(19) United States
(12) Patent Application Publication
(10) Pub. No.: US 2023/0265379 A1
(43) Pub. Date: Aug. 24, 2023

(54) MUSHROOM LINE N-S34, INCORPORATED INTO HYBRID MUSHROOM STRAIN LA3782, AND DERIVATIVES THEREOF

(71) Applicant: Somycel, Langeais (FR)

(72) Inventors: Aniça AMINI, Saint Pierre Des Corps (FR); Sylvie DELBECQUE, Langeais (FR); Stéphanie BITAUDEAU, Mazieres De Touraine (FR); Tomasz KUCZMASZEWSKI, Peterborough (GB); Harry HESEN, Maasbree (NL); Mark WACH, Zillow, PA (US); Mickael O'ROURKE, Navan (IE); Mark LOFTUS, Oakmont, PA (US); Michelle SCHULTZ, New Bethlehem, PA (US); Michael KESSLER, Kittanning, PA (US)

(73) Assignee: Somycel, Langeais (FR)

(21) Appl. No.: 18/018,175

(22) PCT Filed: Jul. 26, 2021
(86) PCT No.: PCT/EP2021/070901

§ 371 Date: Jan. 26, 2023

(30) Foreign Application Priority Data
Jul. 27, 2020 (EP) 20305862.3

(51) Int. Cl. C12N 1/14; A01H 15/00

(52) U.S. Cl. CPC C12N 1/145; A01H 15/00; C12R 2001/645

(57) ABSTRACT

The present invention relates to the development of a homokaryotic Agaricus bisporus (Lange) Imbach mushroom fungus line culture designated N-s34 and to cultures obtained, descended, or otherwise derived from line N-s34. More particularly, the present invention relates to cultures incorporating at least one set of chromosomes having a genotype present in the genotype of the chromosomes found in line N-s34. The present invention further relates to F1 hybrids, and to a particular F1 hybrid strain, designated LA3782, descended from N-s34. This particular strain indeed displays an excellent yield weight of the harvested crop, especially in the third-flush, and a very good shelf-life of the mushroom products. The invention additionally relates to progeny, lines and strains derived from or descended from, or otherwise developed or obtained from, line N-s34 and from said hybrid strain LA3782. The invention further relates to methods of use of the cultures described hereinabove.

### SEQUENCE LISTING

The Sequence Listing file 876090 listing sequence_ST25.txt having a file size of 3000 bytes and a creation date of Jul. 21, 2020, is incorporated herein by reference in its entirety.

### FIELD OF THE INVENTION

The present invention relates to the development of a homokaryotic Agaricus bisporus (Lange) Imbach mushroom fungus line culture designated N-s34 and to cultures obtained, descended, or otherwise derived from line N-s34. More particularly, the present invention relates to cultures incorporating at least one set of chromosomes having a genotype present in the genotype of the chromosomes found in line N-s34. The present invention further relates to F1 hybrids, and to a particular F1 hybrid strain, designated LA3782, descended from N-s34. This particular strain indeed displays an excellent yield weight of the harvested crop, especially in the third-flush, and a very good shelf-life of the mushroom products. The invention additionally relates to progeny, lines and strains derived from or descended from, or otherwise developed or obtained from, line N-s34 and from said hybrid strain LA3782. The invention further relates to methods of use of the cultures described hereinabove.

### BACKGROUND OF THE INVENTION

The edible mushroom Agaricus bisporus (Lange) Imbach var. bisporus, a microorganism belonging to the basidiomycete fungi, is widely cultivated around the world. In Europe and North America, it is the most widely cultivated mushroom species. According to recent market data, “Volume of sales of the 2017-2018 United States mushroom crop totaled 917 million pounds . . . . Value of sales for the 2017-2018 United States mushroom crop was $1.23 billion . . . ” (USDA NASS, 2019). “In 2019, production and sales of cultivated Agaricus bisporus mushrooms in Europe totaled 1,700,000 metric tons, with a market value of approximately 2.5 billion Euros. About 12%, or 200,000 metric tons, of that crop were brown-capped mushrooms.” (Sylvan, Inc, internal market analysis).

Development of novel hybrid mushroom strains or lines of this valuable mushroom fungus is seen as highly desirable to the cultivated mushroom industry, in general to improve genetic diversification of the crop, and particularly if those novel strains or lines can be developed to provide various desirable traits, or novel combinations of traits, within a single strain, culture, hybrid or line.

Cultures are the means by which the mushroom strain developers prepare, maintain, and propagate their industrial microorganisms. Cultures of Agaricus, like those of other microorganisms, are prepared, maintained, propagated and stored on sterile media using various microbiological laboratory methods and techniques known in the art. Sterile tools and aseptic techniques are used within clean rooms or sterile transfer hoods to manipulate cells of pure cultures for various purposes including clonal propagation and for the development of new strains using diverse techniques. Commercial culture inocula including mushroom ‘spawn’ and ‘casing inoculum’ are also prepared using large-scale microbiological production methods, and are provided to the end user as pure cultures on substrate media contained within sterile packaging.

One use of such cultures is to produce mushrooms for sale and consumption. Mushrooms are cultivated commercially within purpose-built structures on dedicated mushroom farms. While there are many variations on methods, and no single standard cultivation method, the following description represents a typical method. Compost prepared from lignocellulosic material such as straw, augmented with nitrogenous material, is finished and pasteurized within a suitable facility. Mushroom spawn, which comprises a sterilized friable ‘carrier substrate’ onto which a pure culture of one mushroom strain has been aseptically incorporated via inoculum and then propagated, is mixed with the pasteurized compost and is incubated for approximately 13 to about 19 days at a controlled temperature, during which time the mycelium of the mushroom culture colonizes the entire mass of compost and begins to digest it. A non-nutritive ‘casing layer’ of material such as peat is then placed over the compost to a depth of from about 1.5 to about 2 inches. Additional ‘casing inoculum’ incorporating the same mushroom culture may be incorporated into the casing layer to accelerate the formation and harvesting of mushrooms, and to improve uniformity of the distribution of mycelium and mushrooms in and on the casing surface. Environmental conditions, including temperature and humidity, within the cropping facility are then carefully managed to promote and control the transition of the culture from vegetative to reproductive growth at the casing/air interface. In a further about 13 to about 18 days after casing, mushrooms will have developed to the correct stage for harvest and sale.

A first flush of mushrooms comprising the original culture will be picked over a 3- to 5-day period. Additional flushes of mushrooms appear at about weekly intervals. Commercially, two or three flushes of mushrooms are produced and harvested before the compost is removed and replaced in the cropping facility. Following harvest, mushrooms are graded, sorted, weighed, packed and shipped under refrigeration. Profitability associated with a strain is dependent upon (1) the yield weight of the harvested crop, net of losses from disease, damage and post-harvest weight loss, (2) variable labor and other costs of harvesting or processing mushrooms of different sizes, weights, spacing/timing behaviors, and types or grades, and (3) crop value based on the quality and marketability of the mushroom product as determined by appearance, physical characteristics, condition during post-harvest storage and marketing (i.e., “shelf life” effects), and market segment (for example white-capped vs. brown-capped, or closed-capped vs. open-capped) of the product.

For many producers, having a steady harvest of mushrooms from day to day or week to week would solve costly problems in harvest- and packing-labor scheduling and management, and also related problems with product inventory, storage and delivery. High yield combined with more balanced yield between flushes is desirable for many growers. While steady production, which is largely a biological trait of individual strains, mitigates some of these costly issues, a further solution to the problem of declines in post-harvest (or ‘shelf-life’) quality and value, including loss of salable weight (due to evaporation and respiration), is desired in the form of a strain which retains more post-harvest weight, or other element of product quality, for a longer time during post-harvest storage.

There is a need for more diverse, more versatile, and more profitable Agaricus bisporus mushroom strains. To meet this need for improved, diverse Agaricus bisporus mushroom strains, various entities within the mushroom industry have set up mushroom strain development programs. The goal of a mushroom strain development program is to combine, in a single strain, culture, hybrid, or line, various desirable traits. Strains currently available to the mushroom industry allow growers to produce crops of mushrooms successfully and profitably. There are many characteristics by which a novel strain might be judged as improved over existing strains, or more suitable, in a particular production facility or sales market, or in the industry regionally or globally. Such characteristics can be assessed using techniques that are well known in the art.

Novel strains are most preferably and successfully developed from new hybridizations (fusions) between haploid homokaryotic lines, including novel lines. Thus, the need continues to exist for new lines that can be used to produce new hybrid strains of Agaricus bisporus mushroom cultures and microorganisms that in turn provide improved and/or novel combinations of characteristics for producer profitability and for improved mushroom products over other previous strains of Agaricus bisporus.

In Assignee's aggregate operating experience of almost 100 years of mushroom strain development in Assignee's research centers, it has been extremely difficult to develop more than a handful of strains which are acceptable with respect to all necessary commercial characteristics. Successful outcomes are rare and generally unpredictable, and rely in part on the serendipitous identification of breeding stocks and lines that are discovered to demonstrate an increased ability or tendency to produce one or more commercially acceptable strains via application of strain development techniques. While many traits could cause a strain not to be commercially acceptable, three of the foremost qualifying traits are crop yield, crop timing, and appearance/“quality” of the mushrooms produced. Therefore, any novel breeding stock or line with the ability to produce acceptable new commercial strains via the application of strain development techniques is of great value to the mushroom strain developer and to the mushroom industry.

Market conditions change over time. Consumer preferences shift and evolve. New pathogens emerge. Raw materials fluctuate in price, composition and availability. Therefore, spawn producers and mushroom producers need access to diversified commercially acceptable strains that present different, alternative combinations of characters that allow for flexible and effective responses to changing market or production conditions, including challenges which may be unforeseeable (e.g., pathogens, agricultural chemical regimens, altered availability or year-to-year properties of particular compost raw materials, etc.). Genetic diversity is responsible for diversity of phenotypic characters including both evident characteristics and others which might not become evident or explicitly valuable except under changed or unpredictable conditions.

Thus, there is a general need for commercially acceptable A. bisporus strains with different, diverse, novel genotypes, relative to other commercially produced strains, for three reasons:

First, strains vegetatively incompatible with other strains in commercial production are known to retard the spread of viral diseases between cultivated strains, due to an inability, or limited ability, of incompatible strains to anastomose (=physically fuse) with each other and exchange cytoplasm. The incompatibility phenotype can be assessed using techniques that are well known in the art. Alternating or rotating the use of incompatible strains within a facility can improve harvest yields immediately, by sharply reducing the transmission/infection rate, while reducing viral disease reservoirs and pressure over a period of weeks or months. Thus there is a need for commercially acceptable mushroom strains that are genetically distinct from and vegetatively incompatible with the other commercial strains now in use, specifically, the brown-capped B14528/Tuscan and BR06/Heirloom strains. The strain B14528 has been deposited under the Budapest Treaty governing the deposit of microorganisms at the Agricultural Research Services Culture Collection (NRRL), Peoria, Ill., USA under NRRL Accession Number 50900. The strain BR06 has been deposited under Budapest Treaty governing the deposit of organisms at the American Type Culture Collection (ATCC), Rockville, Md., USA, under ATCC accession number PTA-6876.

Second, it is well understood that when an agricultural crop industry relies extensively on a single, or only two, genetic lineage(s) (i.e., creates a near-monoculture situation as now exists in most countries for brown-capped mushrooms), there is an increased risk of unpredictable, catastrophic crop failure on a facility-wide or even industry-wide scale, due to emerging diseases or other conditions. Therefore, from a risk management and food security perspective, it is highly desirable to simultaneously provide both genetic diversification and commercially acceptable performance and crop characteristics in an expanded range of commercially available strains.

Third, it is understood that flavor (“taste”) is perceived by different persons in highly individual ways. Both untrained and trained tasters register idiosyncratic preferences for mushrooms produced by different strains; there is no single “best-tasting” mushroom strain, but rather a diverse collection of individual preferences. Preferences for cap color are also diverse and idiosyncratic. Providing genetically diverse offerings of mushrooms provides the consumer with more options and a better chance of finding a mushroom that may become a personal “favorite”. Increased consumer choice and satisfaction supports increased sales pricing and volume and is beneficial to all parties.

Thus, any commercially acceptable hybrid strain, or breeding line, with a novel genotype is useful and advantageous in overcoming the industry-scale problem of limited genetic diversity and global crop resilience, and also the problem of limited options for crop rotation and facility hygiene management, while increasing the prospects for broader consumer acceptance and satisfaction. The use of novel lines that incorporate DNA from non-cultivar stocks meets the need of providing important genetic diversification of the strain pool used to produce crops of cultivated A. bisporus mushrooms. There is an even greater need for diverse and novel breeding lines capable of being used to produce diverse, novel commercially acceptable hybrid strains via strain development techniques. There is a correspondingly great need for the novel hybrid strains so produced in such usage. Every 1% of observed genotypic difference between two strains represents approximately 120 functional genes which may be different.

Most commercial production of brown-capped Agaricus bisporus mushrooms today employs either of only two strains: BR06/Heirloom (PTA-6876) or B14528/Tuscan (NRRL 50900). The mushroom industry has need of other strains that (1) produce an acceptable yield of mushrooms, for example a yield of at least 95%, and preferably of at least 100%, of current commercial strains such as BR06/“Heirloom” or B14528/“Tuscan”, (2) on a desirable commercial production schedule, in other words an harvest schedule that minimizes costs and maximizes crop value, more evenly than the Heirloom strain, while (3) also producing mushrooms of good appearance and high quality for the consumer, and which retain more of their initial weight, compared to the BR06/Heirloom strain, over an extended period of days in the post-harvest sales chain. There is a corresponding need for mushroom lines that can transmit genetic material capable of providing these traits, in addition to other commercially acceptable characteristics, in their hybrid descendent strains.

The present invention fulfills this need by providing new lines and strains that are genetically distinct from all the prior art strains and which meet the desires of mushroom producers, marketers and consumers, including commercially acceptable strains having the specific performance and shelf-life improvements noted above.

### SUMMARY OF THE INVENTION

The present invention is directed generally to an Agaricus bisporus culture comprising at least the set of chromosomes of the Agaricus bisporus line N-s34, a representative culture of said line having been deposited at the Collection Nationale de Cultures de Microorganismes (CNCM), Institut Pasteur, 25 rue du Docteur Roux, 75724 PARIS Cedex 15, on Jun. 30, 2020, under the CNCM Accession Number I-5528, wherein said set of chromosomes comprises preferably the sequence-characterized allelic markers listed in Table I. It is further directed to a culture as described above, characterized in that it is selected from the group consisting of: (a) the line N-s34, a representative culture of same having been deposited under the CNCM Accession Number I-5528 at the Collection Nationale de Cultures de Microorganismes (CNCM), Institut Pasteur, 25 rue du Docteur Roux, 75724 PARIS Cedex 15, on Jun. 30, 2020, and (b) F1 hybrid strains produced by mating the line N-s34 to a second line, and (c) homokaryons of said F1 hybrid strains defined in (b), preferably characterized in that said second line is an homokaryon obtained from strain BP-1, and more preferably characterized in that it is the strain LA3782, a representative culture of said strain having been deposited under the CNCM Accession Number I-5527 at the Collection Nationale de Cultures de Microorganismes (CNCM), Institut Pasteur, 25 rue du Docteur Roux, 75724 PARIS Cedex 15, on Jun. 30, 2020.

Another aspect of the invention relates to an Agaricus bisporus mushroom culture comprising at least one haploid set of chromosomes of the strain LA3782, a representative culture of said strain having been deposited under the CNCM Accession Number I-5527 at the Collection Nationale de Cultures de Microorganismes (CNCM), Institut Pasteur, 25 rue du Docteur Roux, 75724 PARIS Cedex 15, on Jun. 30, 2020, said set of chromosomes preferably comprising the sequence-characterized allelic markers listed in Table II, more preferably characterized in that it is selected from the group consisting of: (a) an homokaryon of the strain LA3782, a representative culture of said strain having been deposited under the CNCM Accession Number I-5527 at the Collection Nationale de Cultures de Microorganismes (CNCM), Institut Pasteur, 25 rue du Docteur Roux, 75724 PARIS Cedex 15, on Jun. 30, 2020, and (b) F2 hybrids produced by mating said homokaryon (a) with a second line.

Another aspect of the invention relates to an Agaricus bisporus mushroom strain culture of the F2, F3, F4, or F5 generation, descended from the F1 hybrid defined above, and preferably from the F1 hybrid LA3782, or from a strain derived from strain LA3782, and comprising respectively at least 40-60%, at least 20-30%, at least 10-15%, or at least 4-8% of the Single-Nucleotide Polymorphisms (SNPs) present in the genome of the Agaricus bisporus strain LA3782, a representative culture of said line having been deposited at the Collection Nationale de Cultures de Microorganismes (CNCM), Institut Pasteur, 25 rue du Docteur Roux, 75724 PARIS Cedex 15, on Jun. 30, 2020, under the CNCM Accession Number I-5527.

Yet another aspect of the invention relates to an Agaricus bisporus mushroom culture that is derived from an initial culture, wherein said initial culture is chosen in the group consisting of: (a) the strain LA3782, a representative culture of said strain having been deposited under the CNCM Accession Number I-5527 at the Collection Nationale de Cultures de Microorganismes (CNCM), Institut Pasteur, 25 rue du Docteur Roux, 75724 PARIS Cedex 15, on Jun. 30, 2020, (b) the Agaricus bisporus line N-s34, a representative culture of said line having been deposited at the Collection Nationale de Cultures de Microorganismes (CNCM), Institut Pasteur, 25 rue du Docteur Roux, 75724 PARIS Cedex 15, on Jun. 30, 2020, under the CNCM Accession Number I-5528, and (c) any culture that is defined hereinabove as a culture of the invention; and which may be characterized in that it comprises at least 10, 20, 30, 40, 50, 60, 70, 80, 90 or at least 100 markers out of the 203 sequence-characterized allelic markers of N-s34 listed in Table 1 or of LA3287 listed in Table 11, or in that it comprises at least 65%, 70%, 75%, 80%, 85%, 90%, 95%, 96%, 97%, 98%, or 99% of the sequence-characterized allelic markers of N-s34 listed in Table 1 or of LA3287 listed in Table II.

In a preferred embodiment of the invention, the culture of the invention as described hereinabove is characterized in that: (a) the yield performance of the crops of said culture are equal to or exceed the yield performance of crops of a BR06/Heirloom strain of Agaricus bisporus, (b) a third-flush yield of the crops of said culture significantly exceeds that of the BR06/Heirloom strain, and (c) the mushroom product of the crops of said culture retains more weight after a number of days of post-harvest storage at 4 degrees Celsius than does the mushroom product of the BR06/Heirloom strain, the number of days selected from the group comprising 3, 4, 5, 6, 7, and 8 days.

The invention also relates to cells, hyphae, mycelium, mushrooms, germinated spores, ungerminated spores, homokaryons, and heterokaryons including SNPs, NSNPs, and aneuploids obtained from a culture of the invention as well as a product incorporating the culture of the invention, including spawn, inoculum, mushrooms, mushroom parts, mushroom pieces, processed foods.

The present invention also relates to a method for developing a new Agaricus bisporus culture, said method comprising applying at least one mushroom strain development technique to the homokaryon line N-s34, a representative culture of which having been deposited at the Collection Nationale de Cultures de Microorganismes (CNCM), Institut Pasteur, 25 rue du Docteur Roux, 75724 PARIS Cedex 15, on Jun. 30, 2020, under the CNCM Accession Number I-5528, or to an homokaryon of the strain LA3782, a representative culture of which having been deposited at the Collection Nationale de Cultures de Microorganismes (CNCM), Institut Pasteur, 25 rue du Docteur Roux, 75724 PARIS Cedex 15, on Jun. 30, 2020, under the CNCM Accession Number I-5527, or to a progeny thereof, to provide a new culture. Preferably, said new culture is characterized in that: (a) the yield performance of the crops of said culture is equal to or exceeds the yield performance of crops of a BR06/Heirloom strain of Agaricus bisporus, (b) a third-flush yield of the crops of said culture significantly exceeds that of the BR06/Heirloom strain, and (c) the mushroom product of the crops of said culture retains more weight after a number of days of post-harvest storage at 4 degrees Celsius than does the mushroom product of the BR06/Heirloom strain, the number of days selected from the group comprising 3, 4, 5, 6, 7, and 8 days. In a preferred embodiment, said new culture is an F2, F3, F4, or F5 hybrid descended from an F1 hybrid of N-s34, or from a strain derived from the strain LA3782, and has a genotype that comprises at least 10, 20, 30, 40, 50, 60, 70, 80, 90 or at least 100 markers out of the 203 sequence-characterized allelic markers of N-s34 listed in Table 1 or of LA3782 listed in Table 11; or has a genotype that comprises at least 40-60%, at least 20-30%, at least 10-15%, or at least 4-8% of the Single-Nucleotide Polymorphisms (SNPs) present in the genome of the Agaricus bisporus line N-s34, a representative culture of said line having been deposited at the Collection Nationale de Cultures de Microorganismes (CNCM), Institut Pasteur, 25 rue du Docteur Roux, 75724 PARIS Cedex 15, on Jun. 30, 2020, under the CNCM Accession Number I-5528.

### DETAILED DESCRIPTION OF THE INVENTION

Genetic identity (e.g., genotype), genealogy, and pedigree are all inextricably interrelated in a strain development or breeding program, as in the cultures of the present invention. The following information on life cycles and heterokaryotic and homokaryotic genotypes, and on parents, offspring, hybrids and descended strains, and derived strains may help to clarify relationships and expectations.

Mushroom-forming fungi exhibit an alternation of generations, from heterokaryotic (N+N, with two haploid nuclei, functionally like the 2N diploid state) to homokaryotic (1N) and further upon mating to become heterokaryotic again. In most eukaryotes, a parent is conventionally considered to be either diploid or heterokaryotic. The haploid ‘generation’ is often, but not always, termed a gamete (e.g., pollen, sperm). In fungi, which are microorganisms, the haploid generation can live and grow indefinitely and independently, for example in laboratory cell culture; while these haploid homokaryons function as gametes in matings, they are equivalent to inbred lines (e.g., of plants) and are more easily referred to as lines (or ‘homokaryon-parents’ of hybrids). Herein, the standalone term ‘parent’ refers, depending on context, to the heterokaryotic culture that is either a, or the, direct progenitor of a haploid line culture, or else the progenitor-once-removed of a strain belonging to the subsequent heterokaryotic generation obtained from a mating of at least one such line. The term ‘line’ thus refers narrowly to a haploid (N) homoallelic culture within the lifecycle. The N+N heterokaryon resulting from a mating, or comprising a breeding stock, or comprising a culture used to produce a crop of mushrooms, may be called a ‘strain’.

Now, with respect to the invention and as noted hereinabove, the present invention relates to at least a homokaryotic line, and more specifically, a culture comprising at least one set of chromosomes of an Agaricus bisporus line designated N-s34, and methods for using the line designated N-s34. The N-s34 line is a homokaryon and its genome and genotype are haploid and thus is entirely homoallelic (although some limited regions of duplicated DNA may be present in its genome).

In a first aspect, the present invention is directed to an Agaricus bisporus culture comprising at least the set of chromosomes of the Agaricus bisporus line N-s34, a representative culture of said line having been deposited at the Collection Nationale de Cultures de Microorganismes (CNCM), Institut Pasteur, rue du Docteur Roux, 75724 PARIS Cedex 15, on Jun. 30, 2020, under the CNCM Accession Number I-5528.

The deposit of a culture of the Agaricus bisporus line N-s34, as disclosed herein, has been made by Somycel, 4 Rue Carnot-ZI Sud, 37130 Langeais, with the Collection Nationale de Cultures de Microorganismes (CNCM). The culture deposited was taken from the same culture maintained by Somycel, Langeais, France, the assignee, since prior to the filing date of this application, and the inventors and assignee have received authorization to refer to this deposited biological material in any and all patent applications. All restrictions upon the deposit have been removed, and the deposit is intended to meet all deposit requirements under the Budapest Treaty. The date of deposit was Jun. 30, 2020. Moreover, the deposit will be maintained in the depository for a period of 30 years, or 5 years after the last request, or for the effective life of any patent, whichever is longer, and will be replaced as necessary during this period. The culture of this deposit will be irrevocably and without restriction of condition released to the public upon the filing of the patent application or upon the issuance of a patent, whichever is required by the applicable patent laws.

Agaricus bisporus mushroom line N-s34 is, based on whole-genome sequencing, a haploid, homokaryotic filamentous basidiomycete culture which in vegetative growth produces a branching network of hyphae, i.e., a mycelium. Growth can produce an essentially two-dimensional colony on the surface of solidified (e.g., agar-based) media, or a three-dimensional mass in liquid or solid-matrix material.

A culture comprising at least one set of chromosomes of an Agaricus bisporus line designated N-s34 may be either a homokaryon or a heterokaryon. It may be (a) line N-s34 itself, (b) a culture having full genotypic identity with N-s34, in agreement with the allelic genotype of N-s34 presented in Table I, (c) a culture having at least one set of genotypic markers which are a subset of those of the genotype of N-s34 representing at least 65%, 70%, 75%, or 80%, 85%, 90%, 95%, 96%, 97%, 98%, 99%, 100%, of the markers present in N-s34, or comprising at least 10, 20, 30, 40, 50, 60, 70, 80, 90 or at least 100 markers out of the 203 sequence-characterized allelic markers of N-s34 listed in Table I (d) a culture having at least 65%, 70%, 75%, 80%, 85%, 90%, 95%, 96%, 97%, 98%, 99%, or 100% genotypic identity with N-s34, and (e) an F1 hybrid having N-s34 as a direct parent, said hybrid displaying all the allelic markers listed in Table I (on at least one of its two alleles).

Cultures of the invention include a culture having at least one genealogical relationship with the culture N-s34, wherein the genealogical relationship is selected from the group of consisting of (1) identity: i.e., self, clone, subculture, (2) descent: i.e., inbred descendent, outbred descendent, back-bred descendent, F1 hybrid, F2 hybrid, F3 hybrid, F4 hybrid, F5 hybrid, and (3) derivation: i.e., derived culture, somatic selection, tissue selection, mutagenized culture, transformed culture. Note that when a relationship involves descent solely from a single parent, the resultant cultures can also be considered to have been ‘derived’ from that parental culture.

In a preferred embodiment, the Agaricus bisporus culture of the invention is selected from the group consisting of:
(a) the line N-s34, a representative culture of same having been deposited under the CNCM Accession Number I-5528 at the Collection Nationale de Cultures de Microorganismes (CNCM), Institut Pasteur, 25 rue du Docteur Roux, 75724 PARIS Cedex 15, on Jun. 30, 2020,
(b) F1 hybrid strains produced by mating the line N-s34 to a second line.

The present invention also targets the homokaryons of said F1 hybrid strains defined in (b), the genome of said homokaryons containing at least 65%, 70%, 75%, 80%, 85%, 90%, 95%, 96%, 97%, 98%, 99%, or 100%, of the markers present in N-s34, among which at least one marker is present in N-s34 but is absent from the second line.

In a preferred embodiment, said second line is an homokaryon obtained from strain BP-1 (also known as AA0096 or ARP023 or PTA-6903).

LA3782 is one example of an F1 hybrid heterokaryon strain having outbred descent from the homokaryotic line N-s34. It is also called Tuscan820. More precisely, it has been obtained by mating the line N-s34 with an homokaryon of strain BP-1 also known as AA-0096 and ARP-023. This strain BP-1 has been deposited under Budapest Treaty governing the deposit of organisms at the American Type Culture Collection (ATCC), Rockville, Md., USA, under ATCC Accession Number PTA-6903.

Mushrooms produced in crops by strain LA3782 are about 39 kg/m² (S.D.±1.98) over 3 flushes in phase 3 system, and typically each weigh about 20-45 grams for medium size mushrooms. Cap color measurements on mushrooms of LA3782 produced L-a-b color of L:71,49 (S.D±2,9) a:7,12 (S.D.±1,02) b:23,28 (S.D.±1,28) when measurements were taken on 30 mushrooms using a Minolta Chromameter.

In a preferred embodiment, the culture of the invention is the strain LA3782, a representative culture of said strain having been deposited under the CNCM Accession Number I-5527 at the Collection Nationale de Cultures de Microorganismes (CNCM), Institut Pasteur, 25 rue du Docteur Roux, 75724 PARIS Cedex 15, on Jun. 30, 2020. The deposit of a culture of the Agaricus bisporus strain LA3782, as disclosed herein, has been made by Somycel, 4 Rue Carnot-ZI Sud, 37130 Langeais, with the Collection Nationale de Cultures de Microorganismes (CNCM). The culture deposited was taken from the same culture maintained by Somycel, Langeais, France, the assignee, since prior to the filing date of this application, and the inventors and assignee have received authorization to refer to this deposited biological material in any and all patent applications. All restrictions upon the deposit have been removed, and the deposit is intended to meet all deposit requirements of the Budapest Treaty. The date of deposit was Jun. 30, 2020. Moreover, the deposit will be maintained in the depository for a period of 30 years, or 5 years after the last request, or for the effective life of any patent, whichever is longer, and will be replaced as necessary during this period. The culture of this deposit will be irrevocably and without restriction of condition released to the public upon the filing of the patent application or upon the issuance of a patent, whichever is required by the applicable patent laws.

Mushroom cultures are most reliably identified by their genotypes, in part because successful cultivar strains are required by the market to conform to a narrow phenotypic range. The genotype can be characterized through a genetic marker profile, which can identify isolates (clones or subcultures) of the same line, strain or culture, or a genealogically related culture including a descendent or a culture derived entirely from an initial culture, or additionally can be used to determine or validate a strain development pedigree over generations.

In Inventor's experience in evaluating whole genome sequences of many dozens of diverse Agaricus bisporus lines and strains, a typical number of SNP markers distinguishing any two unrelated homokaryons is roughly 300,000. This means that transmission of even 1% of a set of chromosomes or genotypic markers in a genealogy still represents about 3,000 distinctive identifying markers for a relationship to N-s34. 200 markers, or even 6 highly polymorphic ones, can establish identity, paternity, and derivation among cultures beyond question, while many thousands of available SNP markers can cumulatively provide a robustly-supported method for establishing genealogical relatedness over multiple generations.

Means of obtaining genetic marker profiles using diverse techniques including whole genome sequencing (WGS) plus Single Nucleotide Polymorphism (SNP) marking and Sequence Characterized Amplified Region (SCAR) marking are well known in the art. Since both approaches can analyze the sequences of specific loci, both provide identical results for any locus (note that in heterokaryon analysis, WGS provides more insight into the distribution of SNPs on the haploid sequences; i.e., confirmation of allelic sequences).

The whole genomic sequence of line N-s34 has been obtained and, consequently, about 95% (about 30.2 Mb) of the entire DNA sequence genotype of line N-s34 is known to the Assignee with certainty. The total number of SNP markers distinguishing the reference genome H97 from line N-s34, and which are known to the Assignee, is at least 141,923. That number is expected to be higher when distinguishing N-s34 from other homokaryons. A brief excerpt of the genotype of line N-s34 and strain LA3782 at numerous sequence-characterized marker loci distributed at intervals along each of the 13 chromosomes of N-s34 and LA3782 is provided in Tables I and II. Only for information, the sequences of the same marker loci are provided for the homokaryotic line J147566s3 disclosed in WO2018/102990.

TABLES I & II
203 SNP marker genotypes for relevant lines and strains

| | | | N-s34 | LA3782 | |
|---|---|---|---|---|---|
| Scaffold ID | Ref Pos | H97 vers 2.0 | Table I | Table II | J147566s3 |
| scaffold_1 | 99995 | CTACATTGA | CTAC [symbol] TTGA | CTAC [symbol] TTGA | CTAC [symbol] TTGA |
| scaffold_1 | 101993 | GAAGGACAT | GAAG [symbol] ACAT | GAAG [symbol] ACAT | GAAG [symbol] ACAT |
| scaffold_1 | 349966 | AAGGTGGTT | AAGG [symbol] GGTT | AAGG [symbol] GGTT | AAGG [symbol] GGTT |
| scaffold_1 | 660050 | TCACCATGA | TCAC [symbol] ATGA | TCAC [symbol] ATGA | TCAC [symbol] ATGA |
| scaffold_1 | 849951 | GATGGAGGA | GATG [symbol] AGGA | GATG [symbol] AGGA | GATG [symbol] AGGA |
| scaffold_1 | 850014 | ATTCCTTTT | ATTC [symbol] TTTT | ATTC [symbol] TTTT | ATTC [symbol] TTTT |
| scaffold-1 | 867820 | GTCACTATT | GTCACTATT | GTCACTATT | GTCACTATT |
| scaffold-1 | 867860 | ATTCTAAAC | ATTC [symbol] AAAC | ATTC [symbol] AAAC | ATTC [symbol] AAAC |
| scaffold-1 | 867868 | CCTTTCCCA | CCTTTCCCA | CCTTTCCCA | CCTTTCCCA |
| scaffold-1 | 867923 | ATCCAGATG | ATCCAGATG | ATCCrGATG | ATCCAGATG |
| scaffold-1 | 867914 | AAAGCATCG | AAAG [symbol] ATCG | AAAG [symbol] ATCG | AAAG [symbol] ATCG |
| scaffold-1 | 867967 | TCAACTGGT | TC [symbol] ACTGGT | TC [symbol] ACTGG [symbol] | TC [symbol] ACTGGT |
| scaffold-1 | 868085 | GGATT--CT | --------- | ggatt--ct | --------- |
| scaffold_1 | 1099971 | GTCGACACC | GTCG [symbol] CACC | GTCG [symbol] CACC | GTCG [symbol] CACC |
| scaffold_1 | 1353901 | AGATAACTA | AGAT [symbol] ACTA | AGAT [symbol] ACTA | AGAT [symbol] ACTA |
| scaffold_1 | 1599956 | AATAAGCGC | AATA [symbol] GCGC | AATA [symbol] GCGC | AATA [symbol] GCGC |
| scaffold_1 | 1850032 | CGAGTAATT | CGAG [symbol] AATT | CGAG [symbol] AATT | CGAG [symbol] AATT |
| scaffold_1 | 2119049 | ACAATCCAA | ACAA [symbol] CAA | ACAA [symbol] CAA | ACAA [symbol] CAA |
| scaffold_1 | 2401751 | CGGATAAAT | CGGATAAAT | CGGA [symbol] AAAT | CGGATAAAT |
| scaffold_1 | 2635654 | TGCGGTTTG | TGCG [symbol] TTTG | TGCG [symbol] TTTG | TGCG [symbol] TTTG |
| scaffold_1 | 2804522 | GAAGACGAC | GAAG [symbol] GAC | GAAG [symbol] GAC | GAAG [symbol] GAC |
| scaffold_1 | 2858975 | GCCGTTCTT | GCCG [symbol] TCTT | GCCG [symbol] TCTT | GCCG [symbol] TCTT |
| scaffold_1 | 3256057 | TATCTGTTT | TATC [symbol] GTTT | TATC [symbol] GTTT | TATC [symbol] GTTT |
| scaffold_2 | 101820 | ATTAAAGAT | ATTAAAGAT | ATTAAAGAT | ATTAAAGAT |
| scaffold_2 | 128192 | TGGACCAGG | T [symbol] GACCAGG | T [symbol] GA [symbol] AGG | T [symbol] GACCAGG |
| scaffold_2 | 279652 | AAGGCATGT | AAGGCATGT | AAGGCATGT | AAGGCATGT |
| scaffold_2 | 350156 | TCGGGGGTG | TCGGGGGTG | TCGG [symbol] GGTG | TCGGGGGTG |
| scaffold_2 | 450323 | CTACCCTTG | CTACCCTTG | CTACCCTTG | CTACCCTTG |
| scaffold_2 | 600112 | ATGTATACG | ATGTATACG | ATGT [symbol] TACG | ATGTATACG |
| scaffold_2 | 850338 | TGGTGCTAA | TGGTGCTAA | TGGT [symbol] CTAA | TGGTGCTAA |
| scaffold_2 | 1099413 | CCTGACTCA | CCTGACTCA | CCTG [symbol] CTCA | CCTGACTCA |
| scaffold_2 | 1189976 | ACGGCCCAA | ACGGCCCAA | ACGG [symbol] CCAA | ACGGCCCAA |
| scaffold_2 | 1293936 | GTGTTTGTT | GTGTTTGTT | GTGT [symbol] TGTT | GTGTTTGTT |
| scaffold_2 | 1349512 | CTCAGCAGT | CTCAGCAGT | CTCA [symbol] CAGT | CTCAGCAGT |
| scaffold_2 | 1378074 | TCCACTTCA | TCCACTTCA | TCCA [symbol] TTCA | TCCACTTCA |
| scaffold_2 | 1378104 | TTTCCAGAT | TTTCCAGAT | TT [symbol] C [symbol] AGAT | TTTCCAGAT |
| scaffold_2 | 1600085 | CACAATGCC | CACAATGCC | CACA [symbol] TGCC | CACAATGCC |
| scaffold_2 | 1643101 | CATCTTCTT | CATC [symbol] TCTT | CATC [symbol] TCTT | CATC [symbol] TCTT |
| scaffold_2 | 1901773 | ACTCGAATT | ACTC [symbol] AATT | ACT [symbol] AATT | ACTC [symbol] AATT |
| scaffold_2 | 2150162 | TGCTTAGGG | TGCTTAGGG | TGCT [symbol] AGGG | TGCTTAGGG |
| scaffold_2 | 2389428 | GGATTTCAA | GGAT [symbol] TCAA | GGAT [symbol] TCAA | GGAT [symbol] TCAA |
| scaffold_2 | 2400281 | TCAAAACCC | TCAA [symbol] AC [symbol] C | [symbol] CAA [symbol] AC [symbol] C | TCAA [symbol] AC [symbol] C |
| scaffold_2 | 2650136 | ATAATTCCT | ATAA [symbol] TCCT | ATAA [symbol] TCCT | ATAATTCCT |
| scaffold_2 | 2904101 | TGTTGAGGT | TGTTGAGGT | TGTT [symbol] AGGT | TGTTGAGGT |
| scaffold_2 | 3049515 | GAAAAGCTT | GAAAAGCTT | GAAA [symbol] GCTT | GAAA [symbol] GCTT |
| scaffold_3 | 57118 | TATAGCAGC | TATAGCAGC | TAT [symbol] CAGC | TAT [symbol] CAGC |
| scaffold_3 | 118150 | GTTTGTCCT | GTTTGTCCT | GTTT [symbol] TCCT | GTTTGTCCT |
| scaffold_3 | 131389 | AGACCGGCG | AGACCGGCG | AGACCGGCG | AGACCGGCG |
| scaffold_3 | 175472 | CTTTATTTC | CTTTATTTC | CTTT [symbol] TTTC | CTTTATTTC |
| scaffold_3 | 250112 | GCAGGAGAG | GCAGGAGAG | GC [symbol] G [symbol] AGAG | GCAGGAGAG |
| scaffold_3 | 379203 | ATAGCGGAA | ATAGCGGAA | ATAG [symbol] GGAA | ATAGCGGAA |
| scaffold_3 | 614937 | CAAAATCTG | CAAAATCTG | CAAA [symbol] TCGT | CAAAATCTG |
| scaffold_3 | 750074 | GTTCTTTTC | GTTCTTTTC | GTTC [symbol] TTTC | GTTCTTTTC |
| scaffold_3 | 1126997 | TCAAAGGCG | TCAAAGGCG | TCAA [symbol] GGCG | TCAAAGGCG |
| scaffold_3 | 1250161 | AGTCTCCTT | AGTCTCCTT | AGTC [symbol] CCTT | AGTCTCCTT |
| scaffold_3 | 1296141 | ATCGGTCAT | ATCGGTCAT | ATCG [symbol] TCAT | ATCGGTCAT |
| scaffold_3 | 1510819 | CCACTGATT | CCACTGATT | CCAC [symbol] GATT | CCACTGATT |
| scaffold_3 | 1774892 | CCGTATGGG | CCGTATGGG | CCGT [symbol] TGGG | CCGTATGGG |
| scaffold_3 | 2008438 | AGCATAGCC | AGCATAGCC | AGCA [symbol] AGCC | AGCATAGCC |
| scaffold_3 | 2250000 | CGTGGCGAT | CGTGGCGAT | CGTGGCGAT | CGTGGCGAT |
| scaffold_3 | 2274053 | AAACCAAGA | AAACCAAGA | AAAC [symbol] AAGA | AAACCAAGA |
| scaffold_3 | 2384173 | TGACCAAGC | TGACCAAGC | TGAC [symbol] AAGC | TGACCAAGC |
| scaffold_3 | 2520748 | TAATTCCAC | TAATTCCAC | TAAT [symbol] CCAC | TAATTCCAC |
| scaffold_3 | 2523207 | CAGTCCATA | CAGTCCATA | CAGT [symbol] ATA | CAGTCCATA |
| scaffold_4 | 100004 | GAGTGATAA | GAGTGATAA | GAGTGATAA | GAGTGATAA |
| scaffold_4 | 460303 | TCCTATAAC | TCCTATAAC | TCCT [symbol] TAAC | TCCTATAAC |
| scaffold_4 | 490648 | CGATCGCGT | CGATCGCGT | CGAT [symbol] GCGT | CGATCGCGT |
| scaffold_4 | 649317 | GAGGCAATG | GAGGCAATG | GAGG [symbol] AAT [symbol] | GAGGCAATG |
| scaffold_4 | 752893 | AAGTCCCAA | AAGTCCCAA | AAGTCCCAA | AAGTCCCAA |
| scaffold_4 | 753018 | TGGGCAAGC | TGGGCAAGC | TGGG [symbol] AAGC | TGGGCAAGC |
| scaffold_4 | 753116 | --------- | --------- | --------- | --------- |
| scaffold_4 | 753134 | AACATAACT | AACATAACT | AACA [symbol] AACT | AACATAACT |
| scaffold_4 | 753165 | TTCC--GAG | TTCC--GAG | TTCC--GAG | TTCC--GAG |
| scaffold_4 | 753221 | CTGTTGGAC | CTGTCGGAC | CTGT [symbol] GGAC | CTGTTGGAC |
| scaffold_4 | 878926 | CTGATCAAT | CTGATCAAT | C [symbol] GA [symbol] CAAT | CTGATCAAT |
| scaffold_4 | 1100085 | GATGCCGAA | GATGCCGAA | GATG [symbol] CGAA | GATGCCGAA |
| scaffold_4 | 1163185 | CAAGCTACT | CAAGCTACT | CAAG [symbol] TACT | CAAGCTACT |
| scaffold_4 | 1350536 | CGAACTCGG | CGAACTCGG | CGAA [symbol] CGG | CGAACTCGG |
| scaffold_4 | 1599885 | GATACTTGC | GATACTTGC | GATACTTGC | GATACTTGC |
| scaffold_4 | 1850288 | ATTCGTGTA | ATTCGTGTA | ATTC [symbol] GTA | ATTCGTGTA |
| scaffold_4 | 1889549 | ACAACAGAA | ACAACAGAA | ACAA [symbol] AGAA | ACAACAGAA |
| scaffold_4 | 2100356 | TCAGAGACC | TCAG [symbol] GACC | TCAG [symbol] GACC | TCAG [symbol] GACC |
| scaffold_4 | 2284257 | TCTGGACTG | TCTG [symbol] ACTG | TCTG [symbol] ACTG | TCTG [symbol] ACTG |
| scaffold_5 | 87962 | GATTAAGGG | GATT [symbol] AGGG | GATT [symbol] AGGG | GATT [symbol] AGGG |
| scaffold_5 | 100211 | TCCTTGAAT | TCCT [symbol] GAAT | TCCT [symbol] GAAT | TCCT [symbol] GAAT |
| scaffold_5 | 350872 | GGCGTGCCC | GGCG [symbol] GCCC | GGCG [symbol] GCCC | GGCG [symbol] GCCC |
| scaffold_5 | 599922 | CGTCATTCA | CGTC [symbol] TTCA | CGTC [symbol] TTCA | CGTC [symbol] TTCA |
| scaffold_5 | 851262 | TAATTCTCT | TAAT [symbol] TCT | TAAT [symbol] TCT | TAAT [symbol] TCT |
| scaffold_5 | 1099776 | ACATTGACA | ACAT [symbol] GACA | ACAT [symbol] GACA | ACAT [symbol] GACA |
| scaffold_5 | 1352539 | TTGTGATCC | TTGT [symbol] TCC | TTGT [symbol] TCC | TTGT [symbol] TCC |
| scaffold_5 | 1599904 | AACTTCCTT | AACT [symbol] CCTT | AACT [symbol] CCTT | AACT [symbol] CCTT |
| scaffold_5 | 1851487 | TTCCGCTCC | TTCCGCTCC | TTCC [symbol] CTCC | TTCCGCTCC |
| scaffold_5 | 2100025 | CCCTTAGTC | CCCT [symbol] AGTC | CCCT [symbol] AGTC | CCCT [symbol] AGTC |
| scaffold_5 | 2278878 | GGTCGAAAA | GGTC [symbol] AAAA | GGTC [symbol] AAAA | GGTC [symbol] AAAA |
| scaffold_6 | 106480 | GCCCACTTG | GCCCACTTG | GCCC [symbol] CTTG | GCCCACTTG |
| scaffold_6 | 350337 | CATTTGGTT | CATTTGGTT | CATT [symbol] GGTT | CATTTGGTT |
| scaffold_6 | 600047 | GGAGCATTT | GGAGCATTT | GGAG [symbol] ATTT | GGAGCATTT |
| scaffold_6 | 849990 | AGTTCAGGA | AGTTCAGGA | AGTT [symbol] AGGA | AGTTCAGGA |
| scaffold_6 | 1098535 | CAAAGATTG | CAAAGATTG | CAAA [symbol] ATTG | CAAAGATTG |
| scaffold_6 | 1349453 | TGTCGGTAG | TGTCGGTAG | TGTC [symbol] TAG | TGTCGGTAG |
| scaffold_6 | 1600000 | AAACCTGGA | AAACCTGGA | AAACCTGGA | AAACCTGGA |
| scaffold_6 | 1764645 | AACCGGATT | AACCGGATT | AACC [symbol] GATT | AACCGGATT |
| scaffold_6 | 2000087 | GATTTTGCG | GATTTTGCG | GATTTTGCG | GATTTTGCG |
| scaffold_6 | 2007502 | AATTGATAA | AATTGATAA | AATT [symbol] ATAA | AATTGATAA |
| scaffold_7 | 100284 | GAAATTCAG | GAAATTCAG | GAAA [symbol] TCAG | GAAATTCAG |
| scaffold_7 | 348994 | CCGGAGTTT | CCGGAGTTT | CCGG [symbol] GTTT | CCGGAGTTT |
| scaffold_7 | 600111 | CAATTATTA | CAATTATTA | CAAT [symbol] ATTA | CAATTATTA |
| scaffold_7 | 850516 | TGACGCATA | TGACGCATA | TGAC [symbol] CATA | TGACGCATA |
| scaffold_7 | 873221 | AATAGACCT | AATAGACCT | AATA [symbol] ACCT | AATAGACCT |
| scaffold_7 | 1100248 | TCACGGAAG | TCACGGAAG | TCAC [symbol] GAAG | TCACGGAAG |
| scaffold_7 | 1352529 | TAAATATAT | TAAATATAT | TAAATATAT | TAAATATAT |
| scaffold_7 | 1605059 | GACAAGCAA | GACAAGCAA | GACA [symbol] GCAA | GACAAGCAA |
| scaffold_7 | 1991524 | CAACCCACC | CAACCCACC | CAAC [symbol] CACC | CAACCCACC |
| scaffold_8 | 350000 | ATTGACGCG | ATTGACGCG | ATTG [symbol] CGCG | ATTG [symbol] CGCG |
| scaffold_8 | 606991 | GTGTATTCT | GTGTATTCT | GTGT [symbol] TTCT | GTGT [symbol] TTCT |
| scaffold_8 | 610549 | GGAACTTGA | GGAACTTGA | GGAA [symbol] TTGA | GGAA [symbol] TTGA |
| scaffold_8 | 829832 | CTGTACAAC | CTGTACAAC | CTGT [symbol] CAAC | CTGTACAAC |
| scaffold_8 | 829846 | TTCGAGTGA | TTCGAGTGA | TTCGAGTGA | TTCGAGTGA |
| scaffold_8 | 830003 | AACTGGCAG | AACTGGCAG | AACTGGCAG | AACT [symbol] GCAG |
| scaffold_8 | 830070 | ATTAGGATT | ATTAGGATT | ATTAGGATT | ATTAGGATT |
| scaffold_8 | 830078 | TACTAGACG | TACTAGACG | TACT [symbol] GACG | TACT [symbol] GACG |
| scaffold_8 | 830105 | ATTTAGCAT | ATTTAGCAT | ATTTAGCAT | ATTTAGCAT |
| scaffold_8 | 830159 | AATTAGAAG | AATTAGAAG | AATTAGAAG | AATTAGAAG |
| scaffold_8 | 830169 | GACGACTGG | GACGACTGG | GACGACTGG | GACGACTGG |
| scaffold_8 | 830215 | AGTGTATCT | AGTGTATCT | AGTG [symbol] ATCT | AGTG [symbol] ATCT |
| scaffold_8 | 830250 | TCCAATGCA | TCCAATGCA | TCCA [symbol] TGCA | TCCA [symbol] TGCA |
| scaffold_8 | 1100000 | CATACGATC | CATACGATC | CATACGATC | CATACGATC |
| scaffold_8 | 1350240 | ACGGGTACT | ACGGGTACT | ACGG [symbol] TACT | ACGGGTACT |
| scaffold_8 | 1354068 | AGAATGCCT | AGAATGCCT | AGAA [symbol] GCCT | AGAA [symbol] GCCT |
| scaffold_8 | 1614036 | TTATCAGTA | TTATCAGTA | TTAT [symbol] AGTA | TTATCAGTA |
| scaffold_8 | 1869238 | TGGAGGTTG | TGGAGGTTG | TGGA [symbol] GTTG | TGGA [symbol] GTTG |
| scaffold_9 | 100447 | CTATTTTCT | CTATTTTCT | CTAT [symbol] TTCT | CTAT [symbol] TTCT |
| scaffold_9 | 350569 | AGAATATAC | AGAA [symbol] ATAC | AGAA [symbol] ATAC | AGAA [symbol] ATAC |
| scaffold_9 | 599950 | TGGTATCCC | T [symbol] GTATCCC | T [symbol] GT [symbol] TCCC | TGGT [symbol] TCCC |
| scaffold_9 | 611788 | TCTGTAATC | T [symbol] TGTAATC | T [symbol] TG [symbol] ATC | T [symbol] TGTAATC |
| scaffold_9 | 721973 | TGTATACGT | TGTA [symbol] ACGT | TGTA [symbol] ACGT | TGTA [symbol] ACGT |
| scaffold_9 | 1010845 | GGGTGGTGA | GGGTGGTGA | G [symbol] GTGGTGA | GGGT [symbol] GTGA |
| scaffold_9 | 1250830 | TTGTGGGGA | TTGT [symbol] GGGA | TTGT [symbol] GGGA | TTGT [symbol] GGGA |
| scaffold_9 | 1499265 | AGTCAGACA | AGTCAGACA | AGTC [symbol] GACA | AGTC [symbol] GACA |
| scaffold_9 | 1499300 | TATGACACC | TATG [symbol] CACC | TATG [symbol] C [symbol] CC | TATGACACC |
| scaffold_9 | 1676755 | CTGCCGTTT | CTGC [symbol] GTTT | CTGC [symbol] GTTT | CTGC [symbol] GTTT |
| scaffold_9 | 1702348 | AGACGCATC | AGACGCATC | AGAC [symbol] CATC | AGAC [symbol] CATC |
| scaffold_9 | 1702552 | CAAAGTCAT | CAAAGTCAT | CAAAGTCAT | CAAAGTCAT |
| scaffold_9 | 1702583 | ACTCAGCTG | ACTCAGCTG | ACTCAGCTG | ACTCAGCTG |
| scaffold_9 | 1702658 | TTGTCGTGG | TTGT [symbol] TGG | TTGT [symbol] TGG | TTGTC [symbol] TGG |
| scaffold_10 | 100470 | TCACCATCG | TCACCATCG | TCAC [symbol] ATCG | TCACCATCG |
| scaffold_10 | 350030 | GCGGCTCAA | GCGGCTCAA | GCGG [symbol] TCAA | GCGG [symbol] TCAA |
| scaffold_10 | 354531 | AATCAATCA | AATCAATCA | AATC [symbol] ATCA | AATC [symbol] ATCA |
| scaffold_10 | 633622 | TGGGCAAAG | TGGGCAAAG | TGGG [symbol] AAAG | TGGG [symbol] AAAG |
| scaffold_10 | 860249 | CCGCAAATT | CCGCAAATT | CCGC [symbol] AATT | CCGCAAATT |
| scaffold_10 | 863401 | ATAAAATTT | ATAAAATTT | ATAAAATTT | ATAAA [symbol] TTT |
| scaffold_10 | 1107782 | CAACCCCAC | CAACCCCAC | CAACCCCAC | CAACCCCAC |
| scaffold_10 | 1338596 | GTGCATCAT | GTGCATCAT | GTGC [symbol] TCAT | GTGC [symbol] TCAT |
| scaffold_10 | 1477092 | AGATGCAAA | AGATGCAAA | AGAT [symbol] CAAA | AGATGCAAA |
| scaffold_10 | 1612161 | TCTTCGGAG | TCTTCGGAG | TCTTCGGAG | TCTTCGGAG |
| scaffold_10 | 1612569 | ATTATATTC | ATTATATTC | ATTATATTC | ATTATATTC |
| scaffold_10 | 1612630 | TGGCTCCTT | TGGCTCCTT | TGGCTCCTT | TGGC [symbol] CCTT |
| scaffold_10 | 1612671 | GGAATCGTC | GGAATCGTC | GGAATCGTC | GGAA [symbol] CGTC |
| scaffold_11 | 101855 | CCAGCCTGT | CCAGCCTGT | CCAG [symbol] CTGT | CCAGCCTGT |
| scaffold_11 | 173230 | AGCGGGCGA | AGCGGGCGA | AGCGGGCGA | AGCGGGCGA |
| scaffold_11 | 350000 | GTCAGCAAG | GTCAGCAAG | GTCAGCAAG | GTCAGCAAG |
| scaffold_11 | 378409 | TGATTGGGG | TGATTGGGG | TGAT [symbol] GGGG | TGATTGGGG |
| scaffold_11 | 600001 | TGGGCGCGC | TGGGCGCGC | TGGG [symbol] GCGC | TGGG [symbol] GCGC |
| scaffold_11 | 627221 | TCTTCGCCC | TCTTCGCCC | TCTT [symbol] GCCC | TCTT [symbol] GCCC |
| scaffold_11 | 929659 | GGAATATCA | GGAATATCA | GGAA [symbol] TCA | GGAATATCA |
| scaffold_11 | 931877 | GACCTCACC | GACCTCACC | GACC [symbol] CACC | GACC [symbol] CACC |
| scaffold_11 | 1155850 | T-TGCCACG | T-TGCCACG | T [symbol] TG [symbol] CACG | T [symbol] T [symbol] CCACG |
| scaffold_11 | 1240230 | ACAAGATTC | ACAAGATTC | ACAA [symbol] ATTC | ACAAGATTC |
| scaffold_11 | 1250447 | GAGGCTACA | GAGGCTACA | GAGG [symbol] TACA | GAGG [symbol] TACA |
| scaffold_12 | 109790 | GTCTGCACC | GTCTGCACC | GTCT [symbol] CACC | GTCTGCACC |
| scaffold_12 | 272255 | CCGAGTGCT | CCGA [symbol] TGCT | CCGA [symbol] TGCT | CCGA [symbol] TGCT |
| scaffold_12 | 281720 | CTTCCGGCG | CTTC [symbol] CG | CTTC [symbol] CG | CTTC [symbol] CG |
| scaffold_12 | 281763 | TCTGCAGCC | TCTGCAGCC | TCTG [symbol] AGCC | TCTGCAGCC |
| scaffold_12 | 554582 | ACTCCGGTC | ACTCCGGTC | ACTC [symbol] GGTC | ACTC [symbol] GGTC |
| scaffold_12 | 770075 | GAACGTTCT | GAAC [symbol] TTCT | GAAC [symbol] TTCT | GAAC [symbol] TTCT |
| scaffold_12 | 909536 | CTATGGAGG | CTATGGAGG | CTAT [symbol] GAGG | CTATGGAGG |
| scaffold_12 | 1000000 | CGAGGAGGA | CGAGGAGGA | CGAG [symbol] AGGA | CGAG [symbol] AGGA |
| scaffold_13 | 100697 | ACGTCTTTA | ACGTCTTTA | ACGTCTTTA | ACGTCTTTA |
| scaffold_13 | 119283 | ACGTTACTG | ACGTTACTG | ACG [symbol] ACTG | ACG [symbol] ACTG |
| scaffold_13 | 363867 | ATCCACTGC | ATCCACTGC | ATCC [symbol] CTGC | ATCC [symbol] CTGC |
| scaffold_13 | 370521 | TTTGAGTCA | TTTGAGTCA | TTTG [symbol] GTCA | TTTG [symbol] GTCA |
| scaffold_13 | 604345 | CTTCAGCAT | CTTCAGCAT | CTTCAGCAT | CTTCAGCAT |
| scaffold_13 | 866136 | GTTGGTCAG | GTTGGTCAG | GTTG [symbol] TCAG | GTTG [symbol] TCAG |
| scaffold_14 | 113109 | AGGGAAATA | AGGGAAATA | AGGG [symbol] AATA | AGGGAAATA |
| scaffold_14 | 372086 | CGATCCCTT | CGATCCCTT | CGAT [symbol] CCTT | CGATCCCTT |
| scaffold_14 | 603118 | GGCCCGCCT | GGCCCGCCT | GGCC [symbol] GCCT | GGCCCGCCT |
| scaffold_14 | 725687 | AGTTCGAAA | AGTTCGAAA | AGTT [symbol] G [symbol] AA | A [symbol] TT [symbol] GAAA |
| scaffold_14 | 808308 | AAGGTATGG | AAGGTATGG | AAG [symbol] ATGG | AAGG [symbol] ATGG |
| scaffold_15 | 101381 | TAAACAGAT | TAAACAGAT | TAAA [symbol] AGAT | TAAA [symbol] AGAT |
| scaffold_15 | 150013 | GTGGCCCGT | GTGGCCCGT | GTGG [symbol] CCGT | GTGGCCCGT |
| scaffold_15 | 367204 | CGCGCCCTA | CGCGCCCTA | CGCG [symbol] CCTA | CGCG [symbol] CCTA |
| scaffold_16 | 106292 | AAGCTGGAA | AAGC [symbol] GGAA | AAGC [symbol] GGAA | AAGC [symbol] GGAA |
| scaffold_16 | 205778 | CAAGGTCTG | CAAG [symbol] TCTG | CAAG [symbol] TCTG | CAAG [symbol] TCTG |
| scaffold_16 | 400000 | CCTCGGATT | CCTCGGATT | CCTCGGATT | CCTCGGATT |
| scaffold_16 | 403998 | CAAAGTACG | CAAAGTACG | CAAA [symbol] TACG | CAAAGTACG |
| scaffold_17 | 134688 | CCCGCTTCA | CCCGCTTCA | CCCG [symbol] TTCA | CCCGCTTCA |
| scaffold_17 | 370858 | GACACAACG | GACA [symbol] AACG | GACA [symbol] AACG | GACA [symbol] AACG |
| scaffold_17 | 449833 | ATCAGACAA | ATCA [symbol] AC [symbol] A | ATCA [symbol] AC [symbol] A | ATCA [symbol] AC [symbol] A |
| scaffold_17 | 472545 | CCGTTCATG | CCGTTCATG | CCGT [symbol] C [symbol] TG | CCGTTCATG |
| scaffold_18 | 112940 | GCGGGTGGG | GCGGGTGGG | GCGG [symbol] TGGG | GCGGGTGGG |
| scaffold_18 | 126322 | CCTCTTCCG | CCTCTTCCG | CCTC [symbol] TCCG | CCTC [symbol] TCCG |
| scaffold_19 | 87323 | CCCAAGCAA | CCCAAGCAA | CCCA [symbol] GCAA | CCCAAGCAA |
| scaffold_19 | 98782 | AAAATTGTT | AAAATTGTT | AAAA [symbol] TGTT | AAAATTGTT |

Tables I and II comprise sets of SNP markers present in N-s34 and LA3782, respectively, described as 9-mers. Positional information refers to the 17 substantial contigs of the H97 V. 2.0 genome sequence assembly (JGI). Because a heterokaryon incorporates two sets of chromosomes, one from each haploid parent, there are two allelic copies (two characters or elements of the genotype) at each marker locus for LA3782. The IUPAC nucleotide and so-called “ambiguity” codes, (also see Annex C, Appendix 2, Table 1, Nucleotide and Amino Acid Symbols as set forth in Standard ST. 25 of the Handbook on Industrial Property Information and Documentation (WIPO) (December 2009)) which are actually heteroallelism codes when used to represent a heterokaryon or diploid genotype, are used in Tables I and II to represent heteroallelic DNA sequence positions, wherein each of two alleles incorporates a different nucleotide at a particular position, in the observed 9-base DNA marker sequences reported above, each of which represents a genotypic marker locus. The identity of each marker locus is specified by the scaffold and SNP position information derived from the H97 V2.0 standard reference genome sequence published by the U.S. Department of Energy Joint Genome Institute (Morin et al. 2012), incorporated herein by reference.

It will be appreciated however that any suitable Polymerase Chain Reaction (PCR) primers that bracket the defined marker regions may be used for identifying the alleles, using methods of designing and using suitable PCR primers that are well known in the art. Distinctions between the homoallelic genotypes of line N-s34 and line H97 are evident, as is the composite nature of the example heteroallelic genotype of F1 hybrid strain LA3782, in which the presence of the genome of N-s34 is evident, as expected, by virtue of perfect conformity, with no conflicts, with the presence of the alleles known to be evident in LA3782. The genotype of strain LA3782 is a composite of those of line N-s34 and the BP-1 homokaryon, and demonstrates that the N-s34 chromosome set can be observed within the F1 hybrid genotype. Methods employing these and other markers to determine genealogical relationships between cultures are provided below.

Alternatively, one can use the six SCAR marker loci p1 n150, ITS, MFPC-1-ELF, AS, AN, and FF as described in U.S. Pat. Nos. 7,608,760, 9,017,988 and below. Each have approximately 10 (or more) known alleles, so that the number of heterokaryotic genotypes possible is on the order of one trillion (1012). These six markers are the six most commonly referenced marker loci in the industry and are considered art standard designations in that all six of the marker loci have been used, in one form or another, to characterize the genotype of Agaricus strains in at least one public source publication. Brief descriptions of relevant alleles at these six unlinked marker loci are provided in Table Ill. Genotypes at these six loci were determined both by Whole Genome Sequencing and by SCAR-PCR, as described in the experimental part below.

TABLE III
allelic markers in the N-s34 line and LA3782 strain of the invention

| Scaffold ID | Ref Pos | H97 vers 2.0 | N-s34 | LA3782 |
|---|---|---|---|---|
| p1n150-G3-2 (scaffold_1) | 868615 | 1T | 2 | 2/5 |
| ITS (scaffold_10) | 1612110 | I1 | I1 | I1/I5 |
| MFPC-ELF (scaffold_8) | 829770 | E1 | E1 | E1/E3 |
| AN (scaffold_9) | 1701712 | N1 | N2 | N2/N3 |
| AS (scaffold_4) | 752867 | SD | SD | SA/SD |
| FF (scaffold_12) | 281674 | FF1 | FF1 | FF1/FF3 |

The markers of Tables I to III can be used for example to empirically determine inclusion of a culture within the scope. Genotype analysis including either Polymerase Chain Reaction (PCR) based analysis of polymorphic regions, or whole genome sequencing, is routinely used to establish the degree and nature of genetic identity with an initial culture to define the class of cultures directly or indirectly derived therefrom in Agaricus bisporus. Either all markers in the derived strain or culture will correspond to markers in the initial strain or culture, or else representation of the markers will typically be higher than 90%, but not lower than 65 or 70%, preferably not lower than 75%, in the derived strain or culture. Using a sufficient number of genetic markers, and especially the 6 highly polymorphic markers of table 1111, the status of a derived strain or culture can be unambiguously determined, and statistically beyond challenge. Similar analyses can establish the nature of the relationship between two cultures, including self, clone, subculture, somatic selection, tissue selection, inbred descendent, outbred descendent, back-bred descendent, transformed culture, mutagenized culture, F1 hybrid, and subsequent generations of hybrids, with high statistical confidence.

In some embodiments, the culture of the invention may be obtained using at least one strain development technique selected from the group consisting of inbreeding, including intramixis, outbreeding, i.e., heteromixis, selfing, backmating, introgressive trait conversion, derivation, somatic selection, tissue selection, single-spore selection, multispore selection, pedigree-assisted breeding, marker assisted selection, mutagenesis and transformation, and applying said at least one strain development technique to a first mushroom culture, or parts thereof, said first culture comprising at least one set of chromosomes of an Agaricus bisporus line N-s34.

If one parental line carries allele ‘p’ at a particular locus, and the other parental line carries allele ‘q’, the F1 hybrid resulting from a mating of these two lines will carry both alleles, and the genotype at that locus can be represented as ‘p/q’ (or ‘pq’, or ‘p+q’). Sequence-characterized markers are ordinarily codominant and both alleles will be evident when an appropriate sequencing protocol is carried out on cellular DNA of the hybrid. After determining the genotypic profile of a strain or hybrid, reference to the genotypic profile of line N-s34 can therefore be used to identify hybrids comprising line N-s34 as a parent, or parental, line, since such hybrids will comprise two sets of alleles, one of which sets will be from, and will match that of, line N-s34. The match can be demonstrated by subtraction of the second allele from the genotype, leaving the N-s34 allele evident at every locus. A refinement of this approach is possible with hybrids of Agaricus bisporus as a consequence of the heterokaryon (N+N) condition existing in hybrids. The two (pre-meiotic, non-recombinant) haploid nuclei can be physically isolated by various known techniques (e.g., protoplasting) into viable ‘neohaplont’ subcultures, and each may then be characterized independently. One of the two neohaplont nuclear genotypes from the F1 hybrid will be that of line N-s34, demonstrating its prior use in the mating step of the method, and its presence in the hybrid. Obtaining deheterokaryotized neohaplont homokaryons from a heterokaryon, including a heterokaryotic culture of the invention, by repartitioning individual haploid nuclei using protoplasting, fragmentation, hyphal tip excision, or other technique, is one method of culture derivation.

As described in the experimental part below, LA3782 has an improved yield, a more balanced yield due to improved third-break yield, and mushrooms with improved keeping qualities, compared to a leading commercial strain, Heirloom/BR06. It achieves these improvements by virtue of a novel genotype which is more than 30% different from other known brown-capped strains (see table VI). That genotype also confers a phenotype that is incompatible with other leading brown-capped strains, providing a barrier to infection by endogenous viruses, a trait which can be exploited by farm hygiene regimens. Further, the genetic distinctness provides genetic diversification of the global mushroom crop, which will provide new opportunities to meet existing and emerging challenges in the diverse markets in which edible Agaricus bisporus mushrooms are grown and sold.

In a preferred embodiment of the invention, the strain culture of the invention is characterized in that the total yield performance of the crops of said culture are equal to or exceed the total yield performance of crops of a BR06/Heirloom or J15051 strain of Agaricus bisporus. Total yield performance can be measured as defined below in large scale trials. During such trials, incubation period can be for example of 18 days in bulk phase III tunnel, spawning rate can be 8 litres/ton of compost phase II. Trays can be filled with 135 kg incubated compost with a filling rate of 90 kg/m². Mc substrate supplement can be added at the rate of 1.33 kg/m². Carbo 9 casing from supplier Euroveen can be applied with 1200 g/m² compost casing, premixed. Airing can start on day 4 after casing. To collect yield, mushrooms can be picked and weighed daily, on at least three replicates. Data can be collected over several flushes. Total yields should be compared on the same number of flushes.

In another embodiment of the invention, the strain culture of the invention is characterized in that the third-flush yield of the crops of said culture significantly exceeds that of the BR06/Heirloom strain. Yield performance can be measured as defined above. Data are preferably collected over at least three flushes. In a preferred embodiment, the third-flush yield of the strain of the invention exceeds the BR06/Heirloom third-flush yield by more than 15%, preferably by more than 20%, more preferably by more than 30% when cultured and picked in the same conditions. The examples below demonstrate that the third-flush yield of LA3782 is also higher than the third-flush yield measured for two other strains of the prior art, namely Tuscan and J15051 (Table VIII). In a preferred embodiment, the third-flush yield of the strain of the invention exceeds the Tuscan and the J15051 third-flush yields by more than 15%, preferably by more than 20%, more preferably by more than 30% when cultured and picked in the same conditions.

In another embodiment, the culture of the invention is a strain of Agaricus bisporus that produces mushrooms which have a significantly higher piece weight than do mushrooms produced by BR06/Heirloom. This trait can be assessed during the first and second flush of mushroom production, on several medium size mushrooms (typically 4-5 cm in diameter). Each replicate is individually weighed. In a preferred embodiment, the mushroom piece weight of the strain of the invention after the first flush exceeds the BR06/Heirloom and Tuscan mushroom piece weight by more than 10%, preferably by more than 20%, more preferably by more than 30% when cultured and picked in the same conditions (Table X below).

In another embodiment, the mushroom product of the crops of said culture retains more weight after a number of days of post-harvest storage at 4 degrees Celsius than does the mushroom product of the BR06/Heirloom strain, the number of days selected from the group comprising 3, 4, 5, 6, 7, and 8 days. This measurement can be done as disclosed in the experimental part below. Piece weight collection can be carried out as disclosed in the example part below. Piece weight is preferably evaluated in Flush 1, for example for three to five replicate styrofoam tills per strain. Briefly, the weight of the empty till is recorded, then a define number mushrooms are placed into each till, spaced enough to not touch each other. They are placed with the stem up, and immediately weighed. This weight corresponds to the “initial weight”. Then the tills are placed at 4° C. for 8 days in a walk-in cooler. The till weights are recorded each day. After subtracting the weight of the empty till, percentage of weight retention can be calculated.

By “significantly”, it is herein meant that the third-flush yield/mushroom weight of the strain of the invention is superior to the yield/mushroom weight of the reference strain with a probability/p-value inferior or equal to 0.05 or less, according to a t-test or other parametric statistical test that compares a series of quantitative results from two or more treatments.

In another embodiment, the strain culture of the invention is able to produce a mushroom whose cap-color is similar to the one of LA3782, as described in Table XI below.

In a preferred embodiment of the invention, the culture of the invention as described hereinabove is characterized in that: (a) the yield performance of the crops of said culture are equal to or exceed the yield performance of crops of a BR06/Heirloom strain of Agaricus bisporus, (b) a third-flush yield of the crops of said culture significantly exceeds that of the BR06/Heirloom strain, and (c) the mushroom product of the crops of said culture retains more weight after a number of days of post-harvest storage at 4 degrees Celsius than does the mushroom product of the BR06/Heirloom strain, the number of days selected from the group comprising 3, 4, 5, 6, 7, and 8 days. The strain BR06/Heirloom is the one that has been deposited under Budapest Treaty governing the deposit of organisms at the American Type Culture Collection (ATCC), Rockville, Md., USA, under ATCC accession number PTA-6876.

Another genetically-determined phenomenon exhibited by Agaricus bisporus and other basidiomycete fungi is vegetative incompatibility. Empirically, it is regularly observed that, in physical contact, a first strain is unable to fuse (anastomose) freely and grow together with any other genetically distinct strain, in other words, with any other strain having less than complete genetic identity with a first strain. The genetics are only partially understood for ‘model’ basidiomycetes, but are known to involve multiple genes and alleles, providing such a large number of combinations that, for practical purposes, each genotype (and each independent strain, including wild strains, cultivars, and hybrids) is extremely unlikely to reoccur in a second strain, and therefore, is effectively unique. The vegetative incompatibility phenotype has two significant commercial and technical implications. First, by using protocols that pair two strains in cropping tests and assess their interaction, it provides a practical test of identity or non-identity between pairs of strains, independent of ‘genetic fingerprinting’. Second, vegetative incompatibility between non-identical strains retards or even prevents the transmission of detrimental viruses between different strains, which can improve facility hygiene and profitability.

In a preferred embodiment of the invention, the culture of the invention is vegetatively incompatible with the strains of the prior art, in particular with the strain BR06/Heirloom or B14528/Tuscan, as shown below.

In a preferred embodiment, the culture of the invention is a culture of a strain of Agaricus bisporus that has less than 99%, 98%, 97%, 96%, 95%, 90%, 80%, 75%, 70%, or 60% genetic similarity to BR06/Heirloom and B14528/Tuscan, and preferably, to a group of any brown-capped strains having both a history of commercial sales and a presence in the record of patent cases in the prior art, the group specifically comprising S600/X618, Bs526, Fr24, Brawn, J15051, BR06/Heirloom and B14528/Tuscan.

In other embodiments, the culture of the invention results from a strain development technique and is a culture derived, descended, or otherwise obtained from the line/strain culture of the invention. The resulting culture thus has at least one genealogical relationship with the initial culture, wherein that genealogical relationship is selected from the group consisting of (1) identity, i.e., self, clone, subculture, (2) descent, i.e., inbred descendent, outcrossed descendent, backcrossed descendent, F1 hybrid, F2 hybrid, F3 hybrid, F4 hybrid, F5 hybrid, and (3) derivation, i.e., derived culture.

LA3782 is an F1 hybrid strain having N-s34 as one parent and a homokaryon from strain BP-1 as a second parent. In strains of the F1 generation incorporating a set of chromosomes and genotypic markers from N-s34, by virtue of direct descent from the N-s34 parent, 50% of the heterokaryotic strain's genotypic markers will be those of the set from N-s34. An F2 hybrid in this genealogy descending from N-s34 will have on average 25%, and typically about 20-30%, of its genotypic markers from those of N-s34. An F3 hybrid in this genealogy descending from N-s34 will have on average 12.5%, and typically about 10-15%, of its genotypic markers from those of N-s34. An F4 hybrid in this genealogy descending from N-s34 will have on average 6.25%, and typically about 4-8%, of its genotypic markers from those of N-s34. An F5 hybrid in this genealogy descending from N-s34 will have on average 3.13%, and typically about 1.5-4.5%, of its genotypic markers from those of N-s34. In other words, the F1 offspring of N-s34 will comprise about 100 out of the 203 sequence-characterized allelic markers of N-s34 listed in Table I, the F2 offspring will comprise about 50 out of the 203 sequence-characterized allelic markers of N-s34 listed in Table I, the F3 offspring of N-s34 will comprise about 25 out of the 203 sequence-characterized allelic markers of N-s34 listed in Table I, and the F4 offspring will comprise about 10 out of the 203 sequence-characterized allelic markers of N-s34 listed in Table I,

The culture of the invention is a strain of Agaricus bisporus that has a genealogical relationship of identity, descent, or derivation from (a) line N-s34 or from (b) strain LA3782. More precisely, the culture of the invention may have, as the initial culture from which it is derived, one of the following cultures: an Agaricus bisporus haploid line culture N-s34, a haploid line culture comprising at least one set of chromosomes of an Agaricus bisporus line N-s34, a hybrid heterokaryotic culture obtained by mating N-s34 with a second culture to produce an F1 generation, any culture of generation F2, F3, F4, F5, inclusive, that is obtained from the F1 generation of the invention, a culture obtained from line N-s34 by using at least one strain development technique, an inbred descendent of N-s34, an outcrossed descendent of N-s34, and a derived variety of any culture that was obtained from N-s34 by using at least one strain development technique.

In a particular aspect, the present invention relates to an Agaricus bisporus mushroom strain culture of the F2, F3, F4, or F5 generation, descended from the F1 hybrid as defined above, and preferably from the F1 hybrid LA3782, or from a strain derived from strain LA3782. Said strain preferably comprises respectively at least 40-60%, at least 20-30%, at least 10-15%, or at least 4-8% of the Single-Nucleotide Polymorphisms (SNPs) present in the genome of the Agaricus bisporus strain LA3782, a representative culture of said line having been deposited at the Collection Nationale de Cultures de Microorganismes (CNCM), Institut Pasteur, 25 rue du Docteur Roux, 75724 PARIS Cedex 15, on Jun. 30, 2020, under the CNCM Accession Number I-5527.

More precisely, the F1 offspring of LA3782 (F2 offspring of N-s34) will comprise at least about 100 allelic markers out of the 203 sequence-characterized allelic markers of LA3782 listed in Table II and at least about 50 allelic markers out of the 203 sequence-characterized allelic markers of N-s34 listed in Table I; the F2 offspring will comprise at least about 50 out of the 203 sequence-characterized allelic markers of LA3782 listed in Table II and at least about 25 out of the 203 sequence-characterized allelic markers of N-s34 listed in Table I; and the F3 offspring of LA3782 will comprise at least about 25 out of the 203 sequence-characterized allelic markers of LA3782 listed in Table II and at least about 10 out of the 203 sequence-characterized allelic markers of N-s34 listed in Table I,

In other words, the strain culture of the invention preferably comprises at least about 100 out of the 203 sequence-characterized allelic markers of LA3782 listed in Table II (F1 offspring of LA3782), at least about 50 out of the 203 sequence-characterized allelic markers of LA3782 listed in Table II (F2 offspring of LA3782) or at least about 25 out of the 203 sequence-characterized allelic markers of LA3782 listed in Table II (F3 offspring of LA3782).

The strain culture of the invention is not the strain BP-1 having been deposited under Budapest Treaty governing the deposit of organisms at the American Type Culture Collection (ATCC), Rockville, Md., USA, under ATCC Accession Number PTA-6903. In a preferred embodiment, the strain culture of the invention differs from BP-1 on at least 10%, 20%, 30%, 40%, or at least 50% of its allelic markers. In other words, the strain culture of the invention does not have more than 90%, 80%, 70%, 60% or 50% of identity with BP-1.

In one embodiment, the culture of the invention has a set of chromosomes having at least 65%, at least 70%, or at least 75% genotypic and genomic identity with the chromosomes of the culture of line N-s34, preferably with the culture of the F1 hybrid produced by mating line N-s34 with a second, different Agaricus bisporus culture, more preferably with the strain LA3782.

In a particular embodiment, the strain of the invention is an F2 hybrid having the F1 hybrid heterokaryon culture LA3782 as at least one parent, and having at least one haploid chromosome set comprising 50% of the allelic markers present in the genotype of the F1 hybrid; an F3 hybrid having said F2 hybrid as at least one parent, and having at least one haploid chromosome set comprising 50% of the allelic markers present in the genotype of the F2 hybrid; an F4 hybrid having said F3 hybrid as at least one parent, and having at least one haploid chromosome set comprising 50% of the allelic markers present in the genotype of the F3 hybrid; an F5 hybrid having said F4 hybrid as at least one parent, and having at least one haploid chromosome set comprising 50% of the allelic markers present in the genotype of the F4 hybrid.

The SNPs present in the genome of the Agaricus bisporus line N-s34 can be easily identified by whole genome sequencing or by using conventional markers such as those described in U.S. Pat. No. 7,608,760 or 9,017,988. Table I gives a number of useful sequences that characterize the line N-s34 of the invention. Any other SNP can however be used to identify progenies of the lines of the invention.

The SNPs present in the genome of the Agaricus bisporus strain LA3782 can be easily identified by whole genome sequencing or by using conventional markers such as those described in U.S. Pat. No. 7,608,760 or 9,017,988. Table II and Table III give a number of useful sequences that characterize the strain LA3782 of the invention. Any other SNP can however be used to identify progenies of the strains of the invention.

In a preferred embodiment, the Agaricus bisporus mushroom strain culture of the invention descends from line N-s34 and contains approximately 50%, approximately 25%, approximately 12.5%, approximately 6.25%, or approximately 3.13% of the SNPs present in the genome of the Agaricus bisporus line N-s34, preferably of the SNPs disclosed in Table I. In another preferred embodiment, the Agaricus bisporus mushroom strain culture of the invention descends from line N-s34 and contains at least about 100, between 50 and 100, between 25 and 50 or between 10 and 25 allelic markers out of the 203 sequence-characterized allelic markers of N-s34 listed in Table I.

In a preferred embodiment, the Agaricus bisporus mushroom strain culture of the invention descends from the strain LA3782 and contains approximately 50%, approximately 25%, approximately 12.5%, approximately 6.25%, or approximately 3.13% of the SNPs present in the genome of the Agaricus bisporus strain LA3782, preferably of the SNPs disclosed in Table II or Table III. In another preferred embodiment, the Agaricus bisporus mushroom strain culture of the invention descends from the strain LA3782 and contains at least about 100, between 50 and 100, between 25 and 50 or between 10 and 25 allelic markers out of the 203 sequence-characterized allelic markers of LA3782 listed in Table II,

The term “approximately” or “about” herein inculcates a range of plus or minus 20% above or below the stated value.

To calculate the percentage of SNPs between two strains, one can compare the composite 9-mer genotype at each locus and assign a value if 1 for a perfect match, or a 0 for anything less than a perfect match. Then the values can be totaled for all loci in each pairwise comparison between strains, and divided by the total number of loci compared. The resulting decimal can be eventually converted to %.

In another embodiment, the culture of the invention comprises at least one set of chromosomes having at least 65%, 70%, 75%, 85%, 90%, 95%, 96%, 97%, 98%, or 99% genetic identity with the chromosomes of N-s34. In a further embodiment, the culture of the invention comprises at least one set of chromosomes having a genotype with at least 65%, 70%, 75%, 85%, 90%, 95%, 96%, 97%, 98%, or 99% representation of the markers present on the chromosomes of N-s34.

More precisely, the Agaricus bisporus mushroom culture of the invention can be derived from the initial culture chosen in the group consisting of:
a) the strain LA3782, a representative culture of said strain having been deposited under the CNCM Accession Number I-5527 at the Collection Nationale de Cultures de Microorganismes (CNCM), Institut Pasteur, 25 rue du Docteur Roux, 75724 PARIS Cedex 15, on Jun. 30, 2020,
b) the Agaricus bisporus line N-s34, a representative culture of said line having been deposited at the Collection Nationale de Cultures de Microorganismes (CNCM), Institut Pasteur, 25 rue du Docteur Roux, 75724 PARIS Cedex 15, on Jun. 30, 2020, under the CNCM Accession Number I-5528, and
c) any culture that is defined above.

Preferably, said culture is characterized in that it comprises at least 65%, 70%, 75%, 80%, 85%, 90%, 95%, 96%, 97%, 98%, 99% or even 100% of the sequence-characterized allelic markers of N-s34 listed in Table I or of LA3782 listed in Table II.

In another aspect, the present invention relates to cells, hyphae, mycelium, mushrooms, germinated spores, ungerminated spores, homokaryons, and heterokaryons including SNPs, NSNPs, and aneuploids obtained from the progeny and derived culture described above.

The present invention also relates to methods for producing the lines and strains of the invention. In particular, the present invention relates to a method for developing a new Agaricus bisporus culture, said method comprising applying at least one mushroom strain development technique to the homokaryon line N-s34, a representative culture of which having been deposited at the Collection Nationale de Cultures de Microorganismes (CNCM), Institut Pasteur, 25 rue du Docteur Roux, 75724 PARIS Cedex 15, on Jun. 30, 2020, under the CNCM Accession Number I-5528 or to a progeny thereof, to provide a new culture.

Also, the present invention relates to a method for developing a new Agaricus bisporus culture, said method comprising applying at least one mushroom strain development technique to an homokaryon of the strain LA3782, a representative culture of which having been deposited at the Collection Nationale de Cultures de Microorganismes (CNCM), Institut Pasteur, 25 rue du Docteur Roux, 75724 PARIS Cedex 15, on Jun. 30, 2020, under the CNCM Accession Number I-5527, or to a progeny thereof, to provide a new culture.

Preferably, said new culture will have any of the features described above for the strains of the invention.

Specifically, said new culture will preferably have any of the following desired traits: (a) an enhanced total yield performance, (b) an enhanced third-flush yield, (c) a good weight, and/or (d) a brown color.

In a preferred embodiment, said new culture will have:
(a) a yield performance of the crops that is equal to or exceeds the yield performance of crops of a BR06/Heirloom strain of Agaricus bisporus, and/or
(b) a third-flush yield of the crops that exceeds that of the BR06/Heirloom strain, and/or
(c) mushroom product of the crops that retains more weight after a number of days of post-harvest storage at 4 degrees Celsius than does the mushroom product of the BR06/Heirloom strain, the number of days selected from the group comprising 3, 4, 5, 6, 7, and 8 days.

These features have been described in details above. The strain BR06/Heirloom is the one that has been deposited under Budapest Treaty governing the deposit of organisms at the American Type Culture Collection (ATCC), Rockville, Md., USA, under ATCC accession number PTA-6876.

In a particularly preferred embodiment, this new culture will be the F2, F3, F4, or F5 generation descended from the F1 hybrid LA3782, or from a strain derived from strain LA3782. As such, it may comprise respectively at least 40-60%, at least 20-30%, at least 10-15%, or at least 4-8% of the Single-Nucleotide Polymorphisms (SNPs) present in the genome of the Agaricus bisporus strain LA3782, a representative culture of said line having been deposited at the Collection Nationale de Cultures de Microorganismes (CNCM), Institut Pasteur, 25 rue du Docteur Roux, 75724 PARIS Cedex 15, on Jun. 30, 2020, under the CNCM Accession Number I-5527.

Preferably, said SNPs are the complete set of SNPs of said hybrid, or a subset thereof, for example the subset disclosed in Table II or Table III.

A number of strain development techniques are known in the art. Some of them are detailed below, in the definition part of the application. Any known technique can be used.

Introducing a desired trait into a culture, for example into Agaricus bisporus line N-s34, can comprise the steps of: (1) physically mating the culture of Agaricus bisporus line N-s34 to a second resultant culture of Agaricus bisporus having the desired trait, to produce a hybrid; (2) obtaining an offspring that carries at least one gene that determines the desired trait from the hybrid; (3) mating said offspring of the hybrid with the culture of Agaricus bisporus line N-s34 to produce a new hybrid; (4) repeating steps (2) and (3) at least once to produce a subsequent hybrid; (5) obtaining a homokaryotic line carrying at least one gene that determines the desired trait and comprising at least 75% of the alleles of line N-s34, for example at the sequence-characterized marker loci described in Table I, from the subsequent hybrid of step (4).

The number “75% of parental DNA in a back-mating (backcross) is an approximation because in the meiosis occurring in the F1 hybrid, random assortment of recombined or unrecombined chromosomes will result in haploid/homokaryotic nuclei having more or less DNA from each of the two parents, balanced around a mean value of 50% (which becomes a mean of 25% in the back-mating).

In another aspect, the present invention relates to a method of producing a mushroom culture comprising the steps of:
(a) growing a progeny culture produced by mating the culture of the invention (typically N-s34 or LA3782) with a second Agaricus bisporus culture;
(b) mating the progeny culture with itself or a different culture to produce a progeny culture of a subsequent generation;
(c) growing a progeny culture of a subsequent generation and mating the progeny culture of a subsequent generation with itself or a different culture; and
(d) repeating steps (b) and (c) for an additional 0-5 generations to produce a mushroom culture.

In a particular embodiment, said method comprises the steps of:
(a) obtaining a molecular marker profile of Agaricus bisporus mushroom line N-s34 or LA3782;
(b) obtaining an F1 hybrid culture comprising at least one set of chromosomes of Agaricus bisporus line N-s34 or of the strain LA3782;
(c) mating a culture obtained from the F1 hybrid culture (b) with a different mushroom culture; and
(d) selecting progeny that possess characteristics of said molecular marker profile of line N-s34 or of strain LA3782.

In another aspect, the present invention relates to a method of producing edible mushrooms, including the step of inoculating compost with a heterokaryotic culture of the invention to produce a crop of mushrooms. A yet further embodiment of the invention is a method of improving farm hygiene, including the step of inoculating compost with the culture of the invention. Yet another embodiment of the invention is a method of crop diversification, including the step of inoculating compost with a culture of the invention.

In another aspect, the present invention also relates to any product incorporating the culture of the invention, including spawn, inoculum, mushrooms, mushroom parts, mushroom pieces, processed foods. All these terms are defined in the “definitions” below.

### Definitions

Initially, in order to provide clear and consistent understanding of the specification and claims, including the scope to be given such terms, the following definitions are provided.
Allele: one of two or more alternative forms of a gene that arise by mutation and are found at the same place on a chromosome; a heritable unit of the genome at a defined locus, ultimately identified by its DNA sequence (or by other means).
Amphithallism: A reproductive syndrome in which heteromixis and intramixis are both active.
Anastomosis: Fusion of two or more hyphae that achieves cytoplasmic continuity.
Basidiomycete: A monophyletic group of fungi producing meiospores on basidia; a member of a corresponding subdivision of Fungi such as the Basidiomycetales or Basidiomycotina.
Basidium: The meiosporangial cell, in which karyogamy and meiosis occur, and upon which the basidiospores are formed.
Bioefficiency: For mushroom crops, the net fresh weight of the harvested crop divided by the dry weight of the compost substrate at the time of spawning, for any given sampled crop area or compost weight.
Breeding: Development of strains, lines or cultures using methods that emphasize sexual mating.
Cap: Pileus; part of the mushroom, the gill-bearing structure.
Cap Roundness: Strictly, a ratio of the maximum distance between the uppermost and lowermost parts of the cap, divided by the maximum distance across the cap, measured on a longitudinally bisected mushroom; typically averaged over many specimens; subjectively, a ‘rounded’ property of the shape of the cap.
Carrier substrate: A medium having both nutritional and physical properties suitable for achieving both growth and dispersal of a culture; examples are substrates that are formulated for mushroom spawn, casing inoculum, and other inoculum.
Casing layer, casing soil, casing: A layer of non-nutritive material such as peat or soil that is applied to the upper surface of a mass of colonized compost in order to permit development of the mushroom crop.
Casing inoculum (CI): A formulation of inoculum material incorporating a mushroom culture, typically of a defined heterokaryotic strain, suitable for mixing into the casing layer.
Cloning: Somatic propagation without selection; produces a clone, which is one category of genealogical relationship (i.e., ‘identity’).
Combining ability: The capacity of an individual to transmit superior performance to its offspring. General combining ability is an average performance of an individual in a particular series of matings.
Compatibility: See heterokaryon compatibility, vegetative compatibility, sexual compatibility; incompatibility is the opposite of compatibility.
Culture: The tangible living organism; the organism propagated on various growth media and substrates; a portion of, or the entirety of, one physical strain, line, homokaryon or heterokaryon; the sum of all of the parts of the culture, including hyphae, mushrooms, spores, cells, protoplasts, nuclei, mitochondria, cytoplasm, DNA, RNA, and proteins, cell membranes and cell walls.
Derivation: Development of strains, lines or cultures generally using methods other than sexual mating, and/or undertake development solely or predominantly from an initial strain or culture; see Derived strain, Derived culture.
Derived culture: A culture obtained by derivation as defined above, exemplified by but not restricted to ‘derived strain’ or ‘derived line’; one category of genealogical relationship.
Derived lineage group: The set of strains or cultures derived solely from a single initial strain or culture (which is the earliest member of the group).
Derived strain/line: A strain/line developed solely or predominantly from a single initial strain/line. Methods employed to obtain derived strains/lines from an initial strain/line include somatic selection, tissue culture selection, single-spore germination, multiple-spore germination, selfing, repeated mating back to the initial culture, mutagenesis, and transformation, to provide some examples. In Agaricus bisporus, properties of derived strains include a high fidelity to the genotype and phenotype of the initial strain. In somatic selection and tissue culture selection, the derived culture may be a clone, or virtually a clone, of the initial culture, and, as with mutagenesis, it may not be feasible to specify an actual difference with the initial strain; measurable genetic identity with the initial strain may reach 100%. In a transformed-derived strain, 99.99+% of the genetic composition is that of the initial strain; the small portion of introduced DNA is ordinarily identifiable. In single-spore germinations and multiple-spore germinations, 100% of the genetic composition of the derived strain is that of the initial strain; however on average about 1% (ranging at about 0-5%) of the initial genetic material may be absent in the derived strain due to recombinational loss of heteroallelism (‘heterozygosity’), thus the ‘derived genotype’ is a subset of the ‘master set’ of the initial strain. In a selfed sibling mating between two compatible haploid homokaryotic offspring from an initial strain, 100% of the genetic composition of the derived strain is that of the initial strain; however on average the loss of initial heteroallelism is about 20%, which is less than the Mendelian expectation of almost 25% due to enforced preservation of heteroallelism on the large Chromosome 1, where the mating compatibility locus MAT is present. Only in a mating of an F1 hybrid back to an initial strain is a substantial portion of the derived genotype, on average about 25%, not present in the initial culture; with repeated back matings to an initial culture, that percentage of non-initial genetic material decreases and approaches zero. When the goal is to preserve a particular trait not present in the initial strain, back-mating may be called ‘single trait conversion.’
Descent: Genealogical descent over a limited number (e.g., 10 or fewer) of sexual generations; one category of genealogical relationship.
Diploid: Having two haploid chromosomal complements within a single nuclear envelope.
Directed mutagenesis: a process of altering the DNA sequence of at least one specific gene locus.
Flesh Thickness: A ratio of the maximum distance between the top of the stem and the uppermost part of the cap, divided by the maximum distance across the cap, measured on a longitudinally bisected mushroom; typically averaged over many specimens; subjectively called ‘meatiness’.

Flush: A period of mushroom production within a cropping cycle, separated by intervals of non-production; the term flush encompasses the terms ‘break’ and ‘wave’ and can be read as either of those terms.
Fungus: A microorganism classified as a member of the Kingdom Fungi.
Genealogical relationship: A familial relationship of identity, descent, or derivation from one or more progenitors, for example that between parents and offspring.
Genetic identity: The genetic information that distinguishes an individual, including representations of said genetic information such as, and including: genotype, genotypic fingerprint, genome sequence, genetic marker profile; “genetically identical”=100% genetic identity, “X % generically identical”=having X % genetic identity, etc. % genetic similarity may be used instead of % generic identity when that percentage is less than 100.
Genotypic fingerprint: A description of the genotype at a defined set of marker loci; the known genotype.
Genetic similarity (or genotypic similarity): an expression of the degree to which one set of genetic markers, i.e., one genotype, resembles another. Any representative set of genetic markers, for example SNP markers, can be used. The proportion of markers shared in the genotypes of two individuals or cultures can be expressed to quantify the degree of resemblance between the two cultures, and is an inversely proportional measure of their distinctiveness. The terms can be used interchangeably with (percent) genetic or genotypic identity. The percentage of similarity can be based on the genotypes for any set of markers.
Gill: Lamella; part of the mushroom, the hymenophore- and basidium-bearing structure.
Haploid: Having only a single complement of nuclear chromosomes; see homokaryon.
Heteroallelic: Having two different alleles at a locus; analogous to heterozygous.
Heteroallelism: Differences between homologous chromosomes in a heterokaryotic genotype;
analogous to heterozygosity.

Heterokaryon: As a term of art this refers to a sexual heterokaryon: a culture which has two complementary (i.e., necessarily heteroallelic at the MAT locus) types of haploid nuclei in a common cytoplasm, and is thus functionally and physiologically analogous to a diploid individual (but cytogenetically represented as N+N rather than 2N), and which is reproductively competent (in the absence of any rare interfering genetic defects at loci other than MAT), and which exhibits vegetative incompatibility reactions with other heterokaryons; also called a strain or stock in the strain development context.
Heterokaryon compatibility: The absence of antagonism observed during physical proximity or contact between two heterokaryons that are not genetically identical; see Heterokaryon Incompatibility.
Heterokaryon incompatibility: The phenomenon of antagonism observed during physical proximity or contact between two heterokaryons that are not genetically identical; a multilocus self/non-self recognition system; i.e., a genetic system that allows one heterokaryon culture to discriminate and recognize another culture as being either self or not-self, that operates in basidiomycete heterokaryons to limit anastomosis (hyphal fusion) and cytoplasmic contact; vegetative incompatibility.
Heterokaryotic: Having the character of a heterokaryon: two haploid nuclei in a common cytoplasm; ordinarily taken to mean two sexually complementary nuclei, but there are exceptions.
Heteromixis: Life cycle involving mating between two different non-sibling haploid individuals or gametes; outbreeding.
Homoallelic: Having not more than one allele at a locus. The equivalent term in a diploid organism is ‘homozygous’. Haploid lines are by definition entirely homoallelic at all non-duplicated loci.
Homokaryon: A haploid culture with a single type (or somatic lineage) of haploid nucleus (cytogenetically represented as N), and which is ordinarily reproductively incompetent, and which does not exhibit typical self/non-self incompatibility reactions with heterokaryons, and which may function as a gamete in sexually complementary anastomoses; a ‘line’ which, as with an inbred plant line, transmits a uniform genotype to offspring; a predominantly homoallelic line that mates well and fruits poorly is a putative homokaryon for strain development purposes; see discussion below.
Homokaryotic: Having the character of a homokaryon; haploid.
Hybrid: Of biparental origin, usually applied to heterokaryotic strains and cultures produced in controlled matings.
Hybridizing: Physical association, for example on a petri dish containing a sterile agar-based nutrient medium, of two cultures, usually homokaryons, in an attempt to achieve anastomosis, plasmogamy, and formation of a sexual heterokaryon (=mating); succeeding in the foregoing.
Hyphae: Threadlike elements of mycelium, composed of cell-like compartments.
Inbreeding: Matings that include sibling-line matings (‘selfing’), back-matings to parent lines or strains, and intramixis; reproduction involving parents that are genetically related.
Induced mutagenesis: a non-spontaneous process of altering the DNA sequence of at least one gene locus.
Initial strain, initial culture: A strain or culture which is used as the sole or predominant starting material in a strain derivation process; more particularly a strain or culture from which a derived strain or derived culture is obtained; the earliest member of a derived lineage group.
Incompatibility: See heterokaryon incompatibility.
Inoculum: A culture in a form that permits transmission and propagation of the culture, for example onto new media; specialized commercial types of inoculum include spawn and CI, wherein the culture is present on a carrier substrate.
Intramixis: A uniparental sexual life cycle involving formation of a complementary ‘mated’ pair of postmeiotic nuclei within the basidium or individual spore; superficially appears to be an asexual process.
Introgressive trait conversion: mating offspring of a hybrid to a parent line or strain such that a desired trait from one strain is introduced into a predominating genetic background of the other parent line or strain.
Lamella: see ‘gill’.
Line: A culture used in matings to produce a hybrid strain; ordinarily a homokaryon which is thus homoallelic, otherwise a non-heterokaryotic (non-NSNPP) culture which is highly homoallelic; practically, a functionally homokaryotic and entirely or predominantly homoallelic culture; analogous in plant breeding to an inbred line which is predominantly or entirely homozygous.
Lineage group: see ‘derived lineage group’. The set of strains or cultures derived solely from a single initial strain or culture.
Locus: A defined contiguous part of the genome, homologous although often varying among different genotypes; plural: loci.
Marker assisted selection: Using linked genetic markers including molecular markers to track trait-determining loci of interest among offspring and through pedigrees.
MAT: The mating-type locus, which determines sexual compatibility and the heterokaryotic state.
Mating: The sexual union of two cultures via anastomosis and plasmogamy; methods of obtaining controlled matings between mushroom cultures are well known in the art.
Mycelium: The vegetative body or thallus of the mushroom organism, comprised of threadlike hyphae.
Mushroom: The reproductive structure of an agaric fungus; an agaric; a cultivated food product of the same name.
Neohaplont: A haploid culture or line obtained by physically deheterokaryotizing (reducing to haploid components) a heterokaryon; a somatically obtained homokaryon; a derived homokaryon.
Offspring: Descendants, for example of a parent heterokaryon, within a single generation; most often used to describe cultures obtained from spores from a mushroom of a strain.
Outbreeding: Mating among unrelated or distantly related individuals.
Parent: An immediate progenitor of an individual; a parent strain is a heterokaryon; a parent line is a homokaryon; a heterokaryon may be the parent of an F1 heterokaryon via an intermediate parent line/homokaryon offspring.
Pedigree-assisted breeding: The use of genealogical information to identify desirable combinations of lines in controlled mating programs.
Phenotype: Observable characteristics of a strain or line as expressed and manifested in an environment.
Plasmogamy: Establishment, via anastomosis, of cytoplasmic continuity leading to the formation of a sexual heterokaryon.
Progenitor: Ancestor, including parent (i.e., the direct progenitor).
Progeny: See Offspring.
Selfing: Mating among sibling lines; see also intramixis.
Sexual compatibility: A condition among different lines having allelic non-identity at the MAT locus, such that two lines are able to mate to produce a stable and reproductively competent heterokaryon. The opposite condition, sexual incompatibility, occurs when two lines each have the same allele at the MAT locus.
Somatic: ‘Of the vegetative mycelium’.
Spawn: A mushroom culture, typically a pure culture of a heterokaryon, typically on a sterile substrate which is friable and dispersible particulate matter, in some instances cereal grain; commercial inoculum for compost; reference to spawn includes reference to the culture on a substrate.
Spore: Part of the mushroom, the reproductive propagule.
Stem: Stipe; part of the mushroom, the cap-supporting structure.
Sterile Growth Media: Nutrient media, sterilized by autoclaving or other methods, that support the growth of the organism; examples include agar-based solid nutrient media such as Potato Dextrose Agar (PDA), nutrient broth, and many other materials.
Stipe: see ‘stem’.
Strain: A heterokaryon with defined characteristics or a specific identity or ancestry.
Targeted mutagenesis: a process of altering the DNA sequence of at least one specific gene locus. Tissue culture: A de-differentiated vegetative mycelium obtained from propagation of a differentiated tissue of the mushroom.
Trait conversion: A method for the selective introduction of the genetic determinants of one (i.e., a single-locus conversion) or more desirable traits into the genetic background of an initial strain while retaining most of the genetic background of the initial strain. See ‘Introgressive trait conversion’ and ‘Transformation’.
Transformation: A process by which the genetic material carried by an individual cell is altered by the incorporation of foreign (exogenous) DNA into its genome or cytoplasm; a method of obtaining a trait conversion including a single-locus conversion, or a novel trait.
Vegetative compatibility: The absence of the phenomenon of antagonism observed during physical proximity or contact between two heterokaryons that are not genetically identical, determined by a multilocus self/non-self recognition system that operates in basidiomycete heterokaryons to limit anastomosis (hyphal fusion) and cytoplasmic contact; Heterokaryon compatibility; the opposite of Vegetative incompatibility.
Vegetative incompatibility: The phenomenon of antagonism observed during physical proximity or contact between two heterokaryons that are not genetically identical, determined by a multilocus self/non-self recognition system that operates in basidiomycete heterokaryons to limit anastomosis (hyphal fusion) and cytoplasmic contact; heterokaryon incompatibility.
Virus-breaking: Using multiple incompatible strains, i.e., strains exhibiting heterokaryon incompatibility, successively in a program of planned strain rotation within a mushroom production facility to reduce the transmission of virus from on-site virus reservoirs into newly planted crops.
Yield: The net fresh weight of the harvest crop, normally expressed in kilograms per square meter.
Yield pattern: The distribution of yield within each flush and among all flushes; influences size, quality, picking costs, and relative disease pressure on the crop and product.

With respect to the definition of homokaryon above, it is noted that homokaryons and homoallelic lines are subject to technical and practical considerations: A homokaryon in classical terms is a haploid culture which is axiomatically entirely homoallelic. In practical terms, for fungal strain development purposes, the definition is broadened somewhat to accommodate both technical limitations and cytological variation, by treating all predominately homoallelic lines as homokaryons. Technical limitations include the fact that genomes contain duplicated DNA regions including repeated elements such as transposons, and may also include large duplications of chromosomal segments due to historical translocation events. Two different A. bisporus genomes sequenced by the Joint Genome Institute (JGI), a U.S. federal facility, differ in estimated length by 4.4%, and in gene numbers by 8.2%, suggesting a considerable amount of DNA duplication or rearrangement within different strains of the species. No presently available genome of A. bisporus can completely account for the physical arrangement of such elements and translocations, and so the assembled genome sequences of haploid lines may have regions that appear to be heteroallelic using currently available genotyping methods. Cytologically, a homokaryotic offspring will ordinarily be a spore that receives one haploid, postmeiotic nucleus. However, a spore receiving two third-division nuclei from the basidium will be genetically equivalent to a homokaryon. A spore receiving two second-division ‘sister’ postmeiotic nuclei will be a functional homokaryon even though some distal ‘islands’ of heteroallelism may be present due to crossovers during meiosis. Also, a meiosis that has an asymmetrical separation of homologues can produce an aneuploid, functionally homokaryotic spore in which an extra chromosome, producing a region of heteroallelism, is present. All of these cultures are highly homoallelic and all function as homokaryons. Technological limitations make it impractical to distinguish among such cultures, and also to rule out DNA segment duplication as an explanation for limited, isolated regions of the genome sequence assembly that appear to be heteroallelic. Therefore, in the present application, the use of the term ‘homoallelic’ to characterize a line includes entirely or predominately homoallelic lines, and cultures described in this way are functional homokaryons, are putatively homokaryotic, and are all defined as homokaryons in the present application.

Agaricus bisporus has a reproductive syndrome known as amphithallism, in which two distinct life cycles, namely heteromixis and intramixis, operate concurrently. As in other fungi, the reproductive propagule is a spore. Agaricus produces spores meiotically, on a meiosporangium known as a basidium. In a first life cycle, A. bisporus spores each receive a single haploid postmeiotic nucleus; these spores are competent to mate but are not competent to produce mushrooms. These haploid spores germinate to produce homokaryotic offspring or lines which can mate with other sexually compatible homokaryons to produce novel hybrid heterokaryons that are competent to produce mushrooms. Heterokaryons generally exhibit much less ability to mate than do homokaryons. This lifecycle is called heteromixis, or more commonly, outbreeding. This life cycle, which may be carried out to obtain new hybrid strains in strain development programs, operates but typically does not predominate in strains of Agaricus bisporus var. bisporus.

A second, inbreeding life cycle called intramixis predominates in most strains of Agaricus bisporus var. bisporus. Most spores, typically 90%-99.9%, receive two post-meiotic nuclei, and most such pairs of nuclei, typically at least 90%, consist of Non-Sister Nuclear Pairs (NSNPs) which have a heteroallelic genotype at most or all centromeric-linked loci including the MAT (=mating type) locus. That MAT genotype determines the expression of the heterokaryotic phenotype of these offspring, which are reproductively competent strains and can produce a crop of mushrooms. Unusually among eukaryotes, relatively lower amounts of chromosomal crossing-over (3.9 crossovers per haploid offspring per generation with the U1 strain as the parent, per Wei Gao, 2014) is observed to have occurred in postmeiotic offspring of Agaricus bisporus; empirically, very little heteroallelism (analogous to heterozygosity), usually not more than 1% on average, per Sonnenberg et al. (2011) is lost among heterokaryotic offspring of a heterokaryotic strain. Consequently, parental and heterokaryotic offspring genotypes and phenotypes tend to closely resemble each other, as noted above. In other words, heterokaryotic offspring of Agaricus bisporus are usually functionally equivalent to, and ordinarily indistinguishable from, their parent, although trivial genetic rearrangements of the parental genome may be present.

A heterokaryotic selfed offspring of an F1 hybrid that itself has a ‘p/q’ genotype at a hypothetical locus will in the example have a genotype of ‘pip’, ‘q/q’, or 134. Two types of selfing lead to differing expectations about representation of alleles of line N-s34 present in the F1 hybrid in the next heterokaryotic generation obtained from a mating of N-s34. When two randomly obtained haploid offspring from the same F1 hybrid, derived from individual spores of different meiotic tetrads, are mated (i.e., in inter-tetrad selfing), representation of the line N-s34 marker profile in each recombined haploid parental line and in each sib-mated heterokaryon will be 50% on average, and slightly more than 75% (to about 85%) of heteroallelism present in the F1 hybrid will on average be retained in the sib-mated heterokaryon (note that the expectation over 75% is due to the mating-compatibility requirement for heteroallelism at the mating type locus (MAT) on the large Chromosome 1, which comprises about 10% of the nuclear genome). Distinctively, in addition, Agaricus bisporus regularly undergoes a second, characteristic, spontaneous intra-tetrad form of selfing called intramixis, producing heterokaryotic postmeiotic spores carrying two different recombined haploid nuclei almost always having complementary, heteroallelic MAT alleles. An offspring developing from any one of these spores is a postmeiotic self-mated heterokaryon with ca. 100% retention of the heteroallelism present in the single F1 parent around all 13 pairs of centromeres. In theory this value would decrease to an average of 66.7% retention of F1 heteroallelism for distal markers unlinked to their centromeres; however empirical observations suggest higher rates of retention even for such distal markers, in conjunction with limited amount of crossing-over. Applicant typically observes 95%-100% retention of heteroallelism in such heterokaryotic offspring; Sonnenberg et al. (2011) reported an average of 99% retention among such offspring of the U1 strain. Transmission of the line N-s34 marker profile in such selfed offspring may be incomplete by a small percentage (typically 0-5%) due to the effects of infrequent meiotic crossovers however while DNA (and genotypic markers) from N-s34 will still represent 50% on average of the resulting heterokaryotic genome. Both types of selfed offspring are considered to be derived strains from the initial F1 hybrid, and the latter type comprises most (often [95-] 99 [-100]%) of the initial genotype of the F1 hybrid, and may express a very similar phenotype to that of the F1 hybrid, and be functionally equivalent to it.

When the relationship is one of inbred descent from a heterokaryon, via offspring homokaryons, the two cultures will have a degree of genetic identity with on average about 85% representation and 100% commonality of origin (with respect to the parental culture). When the relationship is one of intramictic inbred descent from a heterokaryon, via a single heterokaryotic spore, the two cultures will have a degree of genetic identity with on average about 95%-99%-100% representation and 100% commonality of origin (with respect to the parental culture). When the relationship is one of back-bred descent from an F1 heterokaryon, via mating an offspring homokaryon to a parental homokaryon such as N-s34, the representation and commonality of origin of the parental homokaryon genotype in the back-bred heterokaryon will both be roughly 75% on average. Somatic selection cultures and tissue selection cultures will effectively have 100% genetic identity with the initial culture, possibly with epigenetic alterations, or rearrangements, or rare mutations, often present at the same rate as in unselected clonal subcultures, and which are virtually impossible to detect. Mutagenized cultures will similarly have effectively 100% genetic identity with their initial culture, except for one or more random point mutations that are impractical to detect. Transformed cultures will typically have at least 99.99% to 100% genetic identity with their initial culture, plus one small piece of exogenous DNA which may or may not be integrated into the Agaricus genome.

### EXAMPLES

A. Distinction of the Lines/Strains of the Invention from Known Brown Prior Art Strains

The LA3782 strain is substantially different from other brown-capped Agaricus bisporus strains in the prior art.

To demonstrate this, here are provided the allelic genotype data for six standard markers that were previously reported as SCAR markers (U.S. Pat. Nos. 7,608,760, 9,017,988, and subsequent). Brief descriptions of relevant alleles at these six unlinked marker loci are provided below. Genotypes at these six loci were determined both by Whole Genome Sequencing and by SCAR-PCR, as described below.

For the SCAR-PCR method, the amplified PCR product DNA was sequenced by a contractor, Eurofins, using methods of their choice, and the genotypes were determined by direct inspection of these sequences followed by SNP analysis and comparison to Applicant's database of reference marker/allele sequences.

These 6 markers are defined as follows:

The “p1n150-3G-2” marker is a refinement of the p1n150 marker reported on Chromosome 1 by Kerrigan, R. W., et al. “Meiotic behavior and linkage relationships in the secondarily homothallic fungus Agaricus bisporus.” Genetics 133, 225-236 (1993), and shown to be linked to the MAT (mating type) locus by Xu et al., “Localization of the mating type gene in Agaricus bisporus.” App. Env. Microbiol. 59(9): 3044-3049 (1993) and has also been used in other published studies. While several different primers can be and have been used to amplify segments of DNA in which the p1n150-3G-2 marker is present and from which it can be sequenced, digested, electrophoretically characterized, or otherwise analyzed, the primer sequences employed by the inventors for the development of the disclosed data were: Forward: 5′-aggcrycccatcttcasc-3′ (SEQ ID NO:1); Reverse: 5′-gttcgacgacggactgc-3′ (SEQ ID NO:2), with 35 PCR cycles, 56° C. anneal temperature, 1 min. extension time.

The “ITS” marker has been adopted as the official ‘barcode’ sequence for all fungi (Schoch et al., Fungal Barcoding Consortium, “Nuclear ribosomal internal transcribed spacer (ITS) region as a universal DNA barcode marker for Fungi.” Proc. Nat. Acad. Sci. <www.pnas.org/cgi/content/short/1117018109> (2012)), and has been used in innumerable publications, including Morin et al., “Genome sequence of the button mushroom Agaricus bisporus reveals mechanisms governing adaptation to a humic-rich ecological niche.” Proc. Nat'l Acad. Sci. USA 109: 17501-17506 (2012) on the complete A. bisporus genome sequence. White et al. (1990), Amplification and direct sequencing of fungal ribosomal RNA genes for phylogenetics. In: PCR Protocols: a guide to methods and applications. (Innis M A, Gelfand D H, Sninsky J J, White T J, eds). Academic Press, New York, USA: 315-322, published many primer sequences for the ITS marker, of which the inventors used primers ITS1: 5′-TCCGTAGGTGAACCTGCGG-3′ (SEQ ID NO:3) and ITS4: 5′-TCCTCCGCTTATTGATATGC-3′ (SEQ ID NO:4), with 35 PCR cycles, 56° C. anneal temperature, 1 min. extension time.

The “MFPC-1-ELF” marker is derived from a sequence mapped by Marie Foulongne-Oriol et al., “An expanded genetic linkage map of an intervarietal Agaricus bisporus var. bisporus-A. bisporus var. burnettii hybrid based on AFLP, SSR and CAPS markers sheds light on the recombination behaviour of the species.” Fungal Genetics and Biology 47: 226-236 (2010) that is linked to the PPC-1 locus described by Callac et al., “Evidence for PPC1, a determinant of the pilei-pellis color of Agaricus bisporus fruit bodies.” Fungal Genet. Biol. 23, 181-188 (1998). An equivalent linked marker has been used as described in Loftus et al., “Use of SCAR marker for cap color in Agaricus bisporus breeding programs.” Mush. Sci. 15, 201-205 (2000). While several different primers can be and have been used to amplify segments of DNA in which the MFPC-1-ELF marker is present and from which it can be sequenced, digested, electrophoretically characterized, or otherwise analyzed, the primer sequences employed by the inventors for the development of the disclosed data were: Forward: 5′-aytcrcaamaacataccttcaac-3′ (SEQ ID NO:5); reverse: 5′-cattcggcgattttctca-3′ (SEQ ID NO:6), with 35 PCR cycles, 55° C. anneal temperature, 0.5 min. extension time.

The AN, AS, and FF markers were designed from sequences obtained from PCR products produced by the use of primers disclosed by Robles et al., U.S. Pat. No. 7,608,760, and/or from contiguous or overlapping genome sequences, to improve upon the performance, reliability, and consistency of results, as compared to the markers as originally described by Robles et al.; they are genotypically and genomically equivalent. While several different primers can be and have been used to amplify segments of DNA in which either the AN, AS, or FF marker is present and from which it can be sequenced, digested, electrophoretically characterized, or otherwise analyzed, the primer sequences employed by the inventors for the development of the disclosed data were:
AN: Forward: 5′-gacgatgcgggactggtggat-3′ (SEQ ID NO:7); Reverse: 5′-ggtctggcctacrggagtgttgt-3′ (SEQ ID NO:8), with 35 PCR cycles, 64 C anneal temperature, 2 min. extension time.
AS: Forward: 5′-ccgccagcacaaggaatcaaatg-3′ (SEQ ID NO:9); Reverse: 5′-tcagtcggccctcaaaacagtcg-3′ (SEQ ID NO:10), with 35 PCR cycles, 64 C anneal temperature, 2 min. extension time.
FF: Forward: 5′-TCGGGTGGTTGCAACTGAAAAG-3′ ((SEQ ID NO:11); Reverse: TTCCTTTCCGCCTTAATTGTTTCT (SEQ ID NO:12), with 35 PCR cycles, 64° C. anneal temperature, 2 min. extension time.

All the brown strains commercially available in the prior art (Heirloom, Tuscan, S-600, Bs526, Fr24 and Brawn) have been compared to show that the strains of the invention are different. The brown prior art mushroom strain J15051 (NRRL accession number 67316) disclosed in WO2018102290 was also included.

TABLE IV
allelic SCAR markers of various strains

| | | H97 | | | | | | | | | |
|---|---|---|---|---|---|---|---|---|---|---|---|
| | | vers | | | | | | | | | |
| Scaffold ID | Ref Pos | 2.0 | N-s34 | LA3782 | Heirloom | Tuscan | S-600 | Bs526 | Fr 24 | Brawn | J15051 |
| p1n150-G3-2 | 868615 | 1T | 2 | 2/5 | 1T/5 | 2/5 | 1T/2 | 1T/3 | 2/3 | 1T/5 | 2/5 |
| (scaffold_1) | | | | | | | | | | | |
| ITS | 1612110 | I1 | I1 | I1/I5 | I1/I1 | I1/I2 | I2/? | I1/I3 | I3/I4 | I1/I1 | I1/I5 |
| (scaffold_10) | | | | | | | | | | | |
| MFPC-ELF | 829770 | E1 | E1 | E1/E3 | E3/E4 | E3/E4 | E1/E6 | E3/E6 | E2/E7? | E3/E4 | E3/E6 |
| (scaffold_8) | | | | | | | | | | | |
| AN | 1701712 | N1 | N2 | N2/N3 | N2/N3 | N3/N4 | N3/N4 | N4/N4 | N6/N6? | N2/N3 | N3/N4 |
| (scaffold_9) | | | | | | | | | | | |
| AS | 752867 | SD | SD | SA/SD | SA/SD | SC/SD | SC/SD | SC/SD | SC/SD | SB/SD | SA/SD |
| (scaffold_4) | | | | | | | | | | | |
| FF | 281674 | FF1 | FF1 | FF1/FF3 | FF1/FF3 | FF1/FF3 | FF2/FF2 | FF1/FF2 | FF2/FF2 | FF1/FF3 | FF2/FF2 |
| (scaffold_12) | | | | | | | | | | | |

Table IV below summarizes the allelic markers at these 6 loci for the cultures of the invention and for a number of other prior art strains.

Whole-genome sequences were aligned by contigs with reference to the H97 V2.0 reference sequence, using the Seqman NGen module of the Lasergene software package (DNAStar, Inc.). By inspecting the aligned sequences of two or more cultures, SNPs at individual loci have been determined and compared directly.

Tables V and VI below show the genotypes of the relevant strains at the 203 SNP marker loci used in Tables I and II, and also the overall genetic similarity calculation between each strain and LA3782.

TABLE V
Genotypes of LA3782 and six other commercial brown-capped strains (“poor depth” meaning that they
were too few sequence reads to detect the allelic sequence) and of J15051 disclosed in WO2018102290.

| | | | Heirloom/ | Tuscan/ | | | | | |
|---|---|---|---|---|---|---|---|---|---|
| Scaffold | Ref Pos | LA3782 | BR06 | B14528 | S-600 | Bs526 | Fr 24 | Brawn | J15051 |
| scaffold_1 | 99995 | CTAC [symbol] TTGA | CTAC [symbol] TTGA | CTAC [symbol] TTGA | CTAC [symbol] TTGA | CTAC [symbol] TTGA | CTAC [symbol] TTGA | CTAC [symbol] TTGA | CTAC [symbol] TTGA |
| scaffold_1 | 101993 | GAAG [symbol] ACAT | GAAG [symbol] ACAT | GAAG [symbol] ACAT | GAAGGACAT | GAAGGACAT | GAAGGACAT | GAAG [symbol] ACAT | GAAG [symbol] ACAT |
| scaffold_1 | 349966 | AAGG [symbol] GGTT | AAGG [symbol] GGTT | AAGG [symbol] GGTT | AAGG [symbol] GGTT | AAGG [symbol] GGTT | AAGG [symbol] GGTT | AAGG [symbol] GGTT | AAGG [symbol] GGTT |
| scaffold_1 | 660050 | TCAC [symbol] ATGA | TCAC [symbol] ATGA | TCAC [symbol] ATGA | TCAC [symbol] ATGA | TCAC [symbol] ATGA | TCAC [symbol] ATGA | TCAC [symbol] ATGA | TCAC [symbol] ATGA |
| scaffold_1 | 849951 | GATG [symbol] AGGA | GATGGAGGA | GATG [symbol] AGGA | GATG [symbol] AGGA | GAT [symbol] AGGA | GATG [symbol] AGGA | GATGGAGGA | GATG [symbol] AGGA |
| scaffold_1 | 850014 | ATTC [symbol] TTTT | ATTC [symbol] TTTT | ATTC [symbol] TTTT | ATTC [symbol] TTTT | ATTC [symbol] TTTT | ATTC [symbol] TTTT | ATTC [symbol] TTTT | ATTC [symbol] TTTT |
| scaffold_1 | 867820 | GTCACTATT | GTCACTATT | GTCACTATT | GTCACTATT | GTCA [symbol] TATT | GTCA [symbol] TATT | GTCACTATT | GTCACTATT |
| scaffold_1 | 867860 | ATTC [symbol] AAAC | ATTC [symbol] AAAC | ATTC [symbol] AAAC | ATTC [symbol] AAAC | ATTC [symbol] AAAC | ATTC [symbol] AAAC | ATTC [symbol] AAAC | ATTC [symbol] AAAC |
| scaffold_1 | 867868 | CCTTTCCCA | CCTTTCCCA | CCTTTCCCA | CCTTTCCCA | CCTTTCCCA | CCTTTCCCA | CCTTTCCCA | CCTTTCCCA |
| scaffold_1 | 867923 | ATCC [symbol] GATG | ATCC [symbol] GATG | ATCC [symbol] GATG | ATCCAGATG | ATCCAGATG | ATCCAGATG | ATCC [symbol] GATG | ATCC [symbol] GATG |
| scaffold_1 | 867914 | AAAGGATCG | AAAG [symbol] ATCG | AAAG [symbol] ATCG | AAAG [symbol] ATCG | AAAG [symbol] ATCG | AAAG [symbol] ATCG | AAAG [symbol] ATCG | AAAG [symbol] ATCG |
| scaffold_1 | 867967 | TC [symbol] ACTGG [symbol] | TC [symbol] ACTGG [symbol] | TC [symbol] ACTGG [symbol] | TC [symbol] ACTGGT | TCAACTGGT | TC [symbol] ACTGGT | TC [symbol] ACTGG [symbol] | TC [symbol] ACTGG [symbol] |
| scaffold_1 | 868085 | ggatt--ct | ggatt--ct | --------- | ggatt--ct | ggatt--ct | ggatt--ct | ggatt--ct | poor depth |
| scaffold_1 | 1099971 | GTCG [symbol] CACC | GTCGACACC | GTCG [symbol] CACC | GTCG [symbol] CACC | GTCGACACC | GTCG [symbol] CACC | GTCGACACC | GTCG [symbol] CACC |
| scaffold_1 | 1353901 | AGAT [symbol] ACTA | AGAT [symbol] ACTA | AGAT [symbol] ACTA | AGAT [symbol] ACTA | AGAT [symbol] ACTA | AGAT [symbol] ACTA | AGAT [symbol] ACTA | AGAT [symbol] ACTA |
| scaffold_1 | 1599956 | AATA [symbol] GCGC | AATAAGCGC | AATA [symbol] GCGC | AATA [symbol] GCGC | AATAAGCGC | AATAAGCGC | AATAAGCGC | AATA [symbol] GCGC |
| scaffold_1 | 1850032 | CGAG [symbol] AATT | CGAG [symbol] AATT | CGAG [symbol] AATT | CGAG [symbol] AATT | CGAG [symbol] AATT | CGAG [symbol] AATT | CGAG [symbol] AATT | CGAG [symbol] AATT |
| scaffold_1 | 2119049 | ACAA [symbol] CAA | ACAA [symbol] CAA | ACAA [symbol] CAA | ACAA [symbol] CAA | ACAA [symbol] CAA | ACAA [symbol] CAA | ACAA [symbol] CAA | ACAA [symbol] CAA |
| scaffold_1 | 2401751 | CGGA [symbol] AAAT | CGGA [symbol] AAAT | CGGA [symbol] AAAT | CGGATAAAT | CGGA [symbol] AAAT | CGGA [symbol] AAAT | CGGA [symbol] AAAT | CGGA [symbol] AAAT |
| scaffold_1 | 2635654 | TGCG [symbol] TTTG | TGCG [symbol] TTTG | TGCG [symbol] TTTG | TGCG [symbol] TTTG | TGCG [symbol] TTTG | TGCG [symbol] TTTG | TGCG [symbol] TTTG | TGCG [symbol] TTTG |
| scaffold_1 | 2804522 | GAAG [symbol] GAC | GAAG [symbol] GAC | GAAG [symbol] GAC | GAAG [symbol] GAC | GAAG [symbol] GAC | GAAG [symbol] GAC | GAAG [symbol] GAC | GAAG [symbol] GAC |
| scaffold_1 | 2858975 | GCCG [symbol] TCTT | GCCG [symbol] TCTT | GCCG [symbol] TCTT | GCCG [symbol] TCTT | GCCG [symbol] TCTT | GCCG [symbol] TCTT | GCCG [symbol] TCTT | GCCG [symbol] TCTT |
| scaffold_1 | 3256057 | TATC [symbol] GTTT | TATC [symbol] GTTT | TATC [symbol] GTTT | TATC [symbol] GTTT | TATC [symbol] GTTT | TATC [symbol] GTTT | TATC [symbol] GTTT | TATC [symbol] GTTT |
| scaffold_2 | 101820 | ATTAAAGAT | ATTAAAGAT | ATTAAAGAT | ATTAAAGAT | ATTAAAGAT | ATTAAAGAT | ATTAAAGAT | ATTAAAGAT |
| scaffold_2 | 128192 | T [symbol] GA [symbol] AGG | TGGA [symbol] AGG | T [symbol] GA [symbol] AGG | T [symbol] GA [symbol] CAGG | TGGA [symbol] CAGG | TGGACCAGG | TGGA [symbol] AGG | T [symbol] GA [symbol] AGG |
| scaffold_2 | 279652 | AAGGCATGT | AAGG [symbol] ATGT | AAGGCATGT | AAGG [symbol] ATGT | AA [symbol] G [symbol] ATGT | AAGG [symbol] ATGT | AAGG [symbol] ATGT | AAGGCATGT |
| scaffold_2 | 350156 | TCGG [symbol] GGTG | TCGG [symbol] GGTG | TCGG [symbol] GGTG | TCGG [symbol] GGTG | TCGG [symbol] GGTG | TCGG [symbol] GGTG | TCGG [symbol] GGTG | TCGG [symbol] GGTG |
| scaffold_2 | 450323 | CTACCCTTG | CTACCCTTG | CTACCCTTG | CTACCCTTG | CTACCCTTG | CTACCCTTG | CTACCCTTG | CTACCCTTG |
| scaffold_2 | 600112 | ATGT [symbol] TACG | ATGT [symbol] TACG | ATGT [symbol] TACG | ATGT [symbol] TACG | ATGT [symbol] TACG | ATGT [symbol] TACG | ATGT [symbol] TACG | ATGT [symbol] TACG |
| scaffold_2 | 850338 | TGGT [symbol] CTAA | TGGTGCTAA | TGGT [symbol] CTAA | TGGTGCTAA | TGGTGCTAA | TGGTGCTAA | TGGT [symbol] CTAA | TGGT [symbol] CTAA |
| scaffold_2 | 1099413 | CCTG [symbol] CTCA | CCTG [symbol] CTCA | CCTG [symbol] CTCA | CCTG [symbol] CTCA | CCTG [symbol] CTCA | CCTG [symbol] CTCA | CCTG [symbol] CTCA | CCTG [symbol] CTCA |
| scaffold_2 | 1189976 | ACGG [symbol] CCAA | ACGG [symbol] CCAA | ACGG [symbol] CCAA | ACGG [symbol] CCAA | ACGG [symbol] CCAA | ACGG [symbol] CCAA | ACGG [symbol] CCAA | ACGG [symbol] CCAA |
| scaffold_2 | 1293936 | GTGT [symbol] TGTT | GTGT [symbol] TGTT | GTGT [symbol] TGTT | GTGT [symbol] TGTT | GTGT [symbol] TGTT | GTGT [symbol] TGTT | GTGT [symbol] TGTT | GTGT [symbol] TGTT |
| scaffold_2 | 1349512 | CTCA [symbol] CAGT | CTCA [symbol] C [symbol] GT | CTCA [symbol] CAGT | CTCA [symbol] C [symbol] GT | CTCA [symbol] C [symbol] GT | CTCA [symbol] C [symbol] GT | CTCA [symbol] C [symbol] GT | CTCA [symbol] CAGT |
| scaffold_2 | 1378074 | TCCA [symbol] TTCA | TCCA [symbol] TTCA | TCCA [symbol] TTCA | TCCA [symbol] TTCA | TCCA [symbol] TTCA | TCCA [symbol] TTCA | TCCA [symbol] TTCA | TCCA [symbol] TTCA |
| scaffold_2 | 1378104 | TT [symbol] C [symbol] AGAT | TT [symbol] C [symbol] AGAT | TT [symbol] C [symbol] AGAT | TT [symbol] C [symbol] AGAT | TT [symbol] C [symbol] AGAT | TT [symbol] C [symbol] AGAT | TT [symbol] C [symbol] AGAT | TT [symbol] C [symbol] AGAT |
| scaffold_2 | 1600085 | CACA [symbol] TGCC | CACAATGCC | CACA [symbol] TGCC | CACAATGCC | CACAATGCC | CACAATGCC | CACA [symbol] TGCC | CACA [symbol] TGCC |
| scaffold_2 | 1643101 | CATC [symbol] TCTT | CATCTTCTT | CATC [symbol] TCTT | CATC [symbol] TCTT | CATC [symbol] TCTT | CATC [symbol] TCTT | CATC [symbol] TCTT | CATC [symbol] TCTT |
| scaffold_2 | 1901773 | ACT [symbol] AATT | ACTCGAATT | ACT [symbol] AATT | ACTC [symbol] AATT | poor depth | ACTCGAATT | ACT [symbol] AATT | ACT [symbol] AATT |
| scaffold_2 | 2150162 | TGCT [symbol] AGGG | TGCTTAGGG | TGCT [symbol] AGGG | TGCTTAGGG | TGCTTAGGG | TGCTTAGGG | TGCT [symbol] AGGG | TGCT [symbol] AGGG |
| scaffold_2 | 2389428 | GGAT [symbol] TCAA | GGATkTCAA | GGAT [symbol] TCAA | GGAT [symbol] TCAA | GGAT [symbol] TCAA | GGAT [symbol] TCAA | GGAT [symbol] TCAA | GGAT [symbol] TCAA |
| scaffold_2 | 2400281 | [symbol] CAA [symbol] AC [symbol] C | TCAA [symbol] ACCC | [symbol] CAA [symbol] AC [symbol] C | TCAA [symbol] AC [symbol] C | TCAA [symbol] ACCC | TCAA [symbol] ACCC | [symbol] CAA [symbol] ACCC | [symbol] CAA [symbol] AC [symbol] C |
| scaffold_2 | 2650136 | ATAA [symbol] TCCT | ATAATTCCT | ATAA [symbol] TCCT | ATAATTCCT | ATAATTCCT | ATAATTCCT | ATAA [symbol] TCCT | ATAA [symbol] TCCT |
| scaffold_2 | 2904101 | TGTT [symbol] AGGT | TGTTGAGGT | TGTT [symbol] AGGT | TGTTGAGGT | TGTT [symbol] AGGT | TGTTGAGGT | TGTT [symbol] AGGT | TGTT [symbol] AGGT |
| scaffold_2 | 3049515 | GAAA [symbol] GCTT | GAAA [symbol] GCTT | GAAA [symbol] GCTT | GAAA [symbol] GCTT | GAAA [symbol] GCTT | GAAAAGCTT | GAAA [symbol] GCTT | GAAA [symbol] GCTT |
| scaffold_3 | 57118 | TAT [symbol] CAGC | TAT [symbol] CAGC | TATAGCAGC | TAT [symbol] CAGC | TATAGCAGC | TAT [symbol] CAGC | TAT [symbol] CAGC | TAT [symbol] CAGC |
| scaffold_3 | 118150 | GTTT [symbol] TCCT | poor depth | GTTTGTCCT | GTTTGTCCT | GTTTGTCCT | GTTTGTCCT | poor depth | GTTTGTCCT |
| scaffold_3 | 131389 | AGACCGGCG | AGACCGGCG | AGACCGGCG | AGACCGGCG | AGACCGGCG | AGACCGGCG | AGACCGGCG | AGACCGGCG |
| scaffold_3 | 175472 | CTTT [symbol] TTTC | CTTT [symbol] TTTC | CTTT [symbol] TTTC | CTTTATTTC | CTTTATTTC | CTTT [symbol] TTTC | CTTT [symbol] TTTC | CTTTATTTC |
| scaffold_3 | 250112 | GC [symbol] G [symbol] AGAG | GC [symbol] G [symbol] AGAG | GC [symbol] G [symbol] AGAG | GC [symbol] G [symbol] AGAG | GC [symbol] G [symbol] AGAG | GC [symbol] G [symbol] AGAG | GC [symbol] G [symbol] AGAG | GC [symbol] G [symbol] AGAG |
| scaffold_3 | 379203 | ATAG [symbol] GGAA | ATAG [symbol] GGA | ATAG [symbol] GGAA | poor depth | ATAG [symbol] GGAA | ATAG [symbol] GGAA | ATAG [symbol] GGAA | ATAG [symbol] GGAA |
| scaffold_3 | 614937 | CAAA [symbol] TCGT | CAAA [symbol] TCTG | CAAA [symbol] TC [symbol] G | CAAAATCTG | CAAAATCTG | CAAAATCTG | CAAA [symbol] TCTG | CAAAATCTG |
| scaffold_3 | 750074 | GTTC [symbol] TTTC | GTTC [symbol] TTTC | GTTC [symbol] TTTC | GTTC [symbol] TTTC | GTTC [symbol] TTTC | GTTCTTTTC | GTTC [symbol] TTTC | GTTC [symbol] TTTC |
| scaffold_3 | 1126997 | TCAA [symbol] GGCG | TCAA [symbol] GGCG | TCAA [symbol] GGCG | TCAA [symbol] GGCG | TCAA [symbol] GGCG | TCAA [symbol] GGCG | TCAA [symbol] GGCG | TCAA [symbol] GGCG |
| scaffold_3 | 1250161 | AGTC [symbol] CCTT | AGTC [symbol] CCTT | AGTCTCCTT | AGTC [symbol] CCTT | AGTC [symbol] CCTT | AGTC [symbol] CCTT | AGTC [symbol] CCTT | AGTC [symbol] CCTT |
| scaffold_3 | 1296141 | ATCG [symbol] TCAT | ATCG [symbol] TCAT | ATCG [symbol] TCAT | ATCGGTCAT | ATCGGTCAT | ATCGGTCAT | ATCG [symbol] TCAT | ATCGGTCAT |
| scaffold_3 | 1510819 | CCAC [symbol] GATT | CCAC [symbol] GATT | CCAC [symbol] GATT | CCAC [symbol] GATT | CCAC [symbol] GATT | CCAC [symbol] GATT | CCAC [symbol] GATT | CCAC [symbol] GATT |
| scaffold_3 | 1774892 | CCGT [symbol] TGGG | CCGT [symbol] TGGG | CCGT [symbol] TGGG | CCGTATGGG | CCGT [symbol] TGGG | CCGT [symbol] TGGG | CCGT [symbol] TGGG | CCGT [symbol] TGGG |
| scaffold_3 | 2008438 | AGCA [symbol] AGCC | AGCA [symbol] AGCC | AGCA [symbol] AGCC | AGCA [symbol] AGCC | AGCA [symbol] AGCC | AGCA [symbol] AGCC | AGCA [symbol] AGCC | AGCA [symbol] AGCC |
| scaffold_3 | 2250000 | CGTGGCGAT | CGTGGCGAT | CGTGGCGAT | CGTGGCGAT | CGTGGCGAT | CGTGGCGAT | CGTGGCGAT | CGTGGCGAT |
| scaffold_3 | 2274053 | AAAC [symbol] AAGA | AAAC [symbol] AAGA | AAAC [symbol] AAGA | AAAC [symbol] AAGA | AAAC [symbol] AAGA | AAACCAAGA | AAAC [symbol] AAGA | AAAC [symbol] AAGA |
| scaffold_3 | 2384173 | TGAC [symbol] AAGC | TGAC [symbol] AAGC | TGAC [symbol] AAGC | TGAC [symbol] AAGC | TGAC [symbol] AAGC | TGACCAAGC | TGAC [symbol] AAGC | TGACCAAGC |
| scaffold_3 | 2520748 | TAAT [symbol] CCAC | TAAT [symbol] CCAC | TAATTCCAC | TAAT [symbol] CCAC | TAAT [symbol] CCAC | TAATTCCAC | TAAT [symbol] CCAC | TAAT [symbol] CCAC |
| scaffold_3 | 2523207 | CAGT [symbol] ATA | CAGT [symbol] ATA | CAGTCCATA | CAGTCCATA | CAGTCCATA | CAGTCCATA | CAGT [symbol] ATA | CAGT [symbol] ATA |
| scaffold_4 | 100004 | GAGTGATAA | GAGTGATAA | GAGTGATAA | GAGT [symbol] AT [symbol] A | GAGT [symbol] AT [symbol] A | GAGT [symbol] AT [symbol] A | GAGTGATAA | GAGTGATAA |
| scaffold_4 | 460303 | TCCT [symbol] TAAC | TCCT [symbol] TAAC | TCCT [symbol] TAAC | TCCT [symbol] TAAC | TCCT [symbol] TAAC | TCC [symbol] TAAC | TCCT [symbol] TAAC | TCCT [symbol] TAAC |
| scaffold_4 | 490648 | CGAT [symbol] GCGT | CGAT [symbol] GCGT | CGATCGCGT | CGATCGCGT | CGATCGCGT | CGATCGCGT | CGATCGCGT | CGAT [symbol] GCGT |
| scaffold_4 | 649317 | GAGG [symbol] AAT [symbol] | GAGG [symbol] AAT [symbol] | GAGG [symbol] AAT [symbol] | GAGG [symbol] AAT [symbol] | GAGG [symbol] AAT [symbol] | GAGG [symbol] AAT [symbol] | GAGGCAATG | GAGG [symbol] AAT [symbol] |
| scaffold_4 | 752893 | AAGTCCCAA | AAGTCCCAA | AAGT [symbol] CCAA | AAGT [symbol] CCAA | AAGT [symbol] CCAA | AAGT [symbol] CCAA | AAGTCCCAA | AAGTCCCAA |
| scaffold_4 | 753018 | TGGG [symbol] AAGC | TGGG [symbol] AAGC | TGGG [symbol] AAGC | TGGG [symbol] AAGC | TGGG [symbol] AAGC | TGGG [symbol] AAGC | TGGG [symbol] AAGC | TGGG [symbol] AAGC |
| scaffold_4 | 753116 | ------ | ------ | gatatc | gatatc | gatatc | GATATC | ------ | ---- |
| scaffold_4 | 753134 | AACA [symbol] AACT | AACA [symbol] AACT | AACA [symbol] AACT | AACA [symbol] AACT | AACA [symbol] AACT | AACA [symbol] AACT | AACATAACT | AACA [symbol] AACT |
| scaffold_4 | 753165 | TTCC--GAG | TTCC--GAG | TTCC--GAG | TTCC--GAG | TTCC--GAG | TTCC--GAG | TTCC--GAG | TTCC--GAG |
| scaffold_4 | 753221 | CTGT [symbol] GGAC | CTGT [symbol] GGAC | CTGT [symbol] GGAC | CTGT [symbol] GGAC | CTGT [symbol] GGAC | CTGT [symbol] GGAC | CTGT [symbol] GGAC | CTGT [symbol] GGAC |
| scaffold_4 | 878926 | C [symbol] GA [symbol] CAAT | C [symbol] GA [symbol] CAAT | C [symbol] GA [symbol] CAAT | C [symbol] GA [symbol] CAAT | C [symbol] GA [symbol] CAAT | C [symbol] GA [symbol] CAAT | C [symbol] GA [symbol] CAAT | C [symbol] GA [symbol] CAAT |
| scaffold_4 | 1100085 | GATG [symbol] CGAA | GATG [symbol] CGAA | GATG [symbol] CGAA | GATG [symbol] CGAA | GATG [symbol] CGAA | GATG [symbol] CGAA | GATG [symbol] CGAA | GATG [symbol] CGAA |
| scaffold_4 | 1163185 | CAAG [symbol] TACT | CAAG [symbol] TACT | CAA [symbol] TACT | CAA [symbol] TACT | CAA [symbol] TACT | CAA [symbol] TACT | CAA [symbol] TACT | CAAG [symbol] TACT |
| scaffold_4 | 1350536 | CGAA [symbol] CGG | CGAA [symbol] CGG | CGAA [symbol] CGG | CGAA [symbol] CGG | CGAA [symbol] CGG | CGAA [symbol] CGG | CGAA [symbol] CGG | CGAA [symbol] CGG |
| scaffold_4 | 1599885 | GATACTTGC | GATACTTGC | GATA [symbol] TTGC | GATA [symbol] TTGC | GATA [symbol] TTGC | GATA [symbol] TTGC | GATACTTGC | GATACTTGC |
| scaffold_4 | 1850288 | ATTC [symbol] GTA | ATTC [symbol] GTA | ATTC [symbol] GTA | ATTC [symbol] GTA | ATTC [symbol] GTA | ATTC [symbol] GTA | ATTC [symbol] GTA | ATTC [symbol] GTA |
| scaffold_4 | 1889549 | ACAA [symbol] AGAA | ACAA [symbol] AGAA | ACAA [symbol] AGAA | ACAACAGAA | ACAA [symbol] AGAA | ACAA [symbol] AGAA | ACAA [symbol] AGAA | ACAA [symbol] AGAA |
| scaffold_4 | 2100356 | TCAG [symbol] GACC | TCAG [symbol] GACC | TCAGAGACC | poor depth | TCAGAGACC | TCAG [symbol] GACC | TCAG [symbol] GACC | TCAG [symbol] GACC |
| scaffold_4 | 2284257 | TCTG [symbol] ACTG | TCTG [symbol] ACTG | TCTGGACTG | TCTG [symbol] ACTG | TCTGGACTG | TCTG [symbol] ACTG | TCTG [symbol] ACTG | TCTG [symbol] ACTG |
| scaffold_5 | 87962 | GATT [symbol] AGGG | GATT [symbol] AGGG | GATT [symbol] AGGG | GATT [symbol] AGGG | GATT [symbol] AGGG | GATT [symbol] AGGG | GATT [symbol] AGGG | GATT [symbol] AGGG |
| scaffold_5 | 100211 | TCCT [symbol] GAAT | TCCT [symbol] GAAT | TCCT [symbol] GAAT | poor depth | TCCT [symbol] GAAT | TCCT [symbol] GAAT | TCCT [symbol] GAAT | TCCT [symbol] GAAT |
| scaffold_5 | 350872 | GGCG [symbol] GCCC | GGCG [symbol] GCCC | GGCGTGCCC | GGCG [symbol] GCCC | GGCGTGCCC | GGCGTGCCC | GGCG [symbol] GCCC | GGCG [symbol] GCCC |
| scaffold_5 | 599922 | CGTC [symbol] TTCA | CGTC [symbol] TTCA | CGTCATTCA | CGTC [symbol] TTCA | CGTCATTCA | CGTCATTCA | CGTC [symbol] TTCA | CGTC [symbol] TTCA |
| scaffold_5 | 851262 | TAAT [symbol] TCT | TAAT [symbol] TCT | TAAT [symbol] TCT | TAAT [symbol] TCT | TAAT [symbol] TCT | TAA [symbol] TCT | TAAT [symbol] TCT | TAAT [symbol] TCT |
| scaffold_5 | 1099776 | ACAT [symbol] GACA | ACAT [symbol] GACA | ACAT [symbol] GACA | poor depth | ACAT [symbol] GACA | ACAT [symbol] GACA | ACAT [symbol] GACA | ACAT [symbol] GACA |
| scaffold_5 | 1352539 | TTGT [symbol] TCC | TTGT [symbol] TCC | TTGT [symbol] TCC | TTGT [symbol] TCC | TTGT [symbol] TCC | TTGT [symbol] TCC | TTGT [symbol] TCC | TTGT [symbol] TCC |
| scaffold_5 | 1599904 | AACT [symbol] CCTT | AACT [symbol] CCTT | AACT [symbol] CCTT | poor depth | AACT [symbol] CCTT | AACT [symbol] CCTT | AACT [symbol] CCTT | AACT [symbol] CCTT |
| scaffold_5 | 1851487 | TTCC [symbol] CTCC | TTCCGCTCC | TTCCGCTCC | poor depth | TTCCGCTCC | TTCC [symbol] CTCC | TTCC [symbol] CTCC | TTCC [symbol] CTCC |
| scaffold_5 | 2100025 | CCCT [symbol] AGTC | CCCT [symbol] AGTC | CCCT [symbol] AGTC | poor depth | CCCT [symbol] AGTC | CCCT [symbol] AGTC | CCCT [symbol] AGTC | CCCT [symbol] AGTC |
| scaffold_5 | 2278878 | GGTC [symbol] AAAA | GGTC [symbol] AAAA | GGTCGAAAA | GGTC [symbol] AAAA | GGTCGAAAA | GGTCGAAAA | GGTC [symbol] AAAA | GGTC [symbol] AAAA |
| scaffold_6 | 106480 | GCCC [symbol] CTTG | GCCC [symbol] CTTG | GCCC [symbol] CTTG | GCCC [symbol] CTTG | GCCC [symbol] CTTG | GCCC [symbol] CTTG | GCCC [symbol] CTTG | GCCC [symbol] CTTG |
| scaffold_6 | 350337 | CATT [symbol] GGTT | CATT [symbol] GGTT | CATT [symbol] GGTT | CATT [symbol] GGTT | CATT [symbol] GGTT | CATTTGGTT | CATT [symbol] GGTT | CATT [symbol] GGTT |
| scaffold_6 | 600047 | GGAG [symbol] ATTT | GGAG [symbol] ATTT | GGAG [symbol] ATTT | GGAG [symbol] ATTT | GGAG [symbol] ATTT | GGAGCATTT | GGAGCATTT | GGAG [symbol] ATTT |
| scaffold_6 | 849990 | AGTT [symbol] AGGA | AGTT [symbol] AGGA | AGTT [symbol] AGGA | AGTT [symbol] AGGA | AGTT [symbol] AGGA | AGTTCAGGA | AGTTCAGGA | AGTT [symbol] AGGA |
| scaffold_6 | 1098535 | CAAA [symbol] ATTG | CAAA [symbol] ATTG | CAAA [symbol] ATTG | CAAA [symbol] ATTG | CAAA [symbol] ATTG | CAAA [symbol] ATTG | CAAA [symbol] ATTG | CAAA [symbol] ATTG |
| scaffold_6 | 1349453 | TGTC [symbol] TAG | TGTC [symbol] TAG | TGTC [symbol] TAG | TGTC [symbol] TAG | TGTC [symbol] TAG | TGTCGGTAG | TGTCGGTAG | TGTC [symbol] TAG |
| scaffold_6 | 1600000 | AAACCTGGA | AAACCTGGA | AAACCTGGA | AAACCTGGA | AAACCTGGA | AAACCTGGA | AAA [symbol] TGGA | AAACCTGGA |
| scaffold_6 | 1764645 | AACC [symbol] GATT | AACC [symbol] GATT | AACC [symbol] GATT | AACC [symbol] GATT | AACC [symbol] GATT | AACC [symbol] GATT | AACC [symbol] GATT | AACC [symbol] GATT |
| scaffold_6 | 2000087 | GATTTTGCG | GATT [symbol] TGCG | GATT [symbol] TGCG | poor depth | poor depth | GATT [symbol] TGCG | GATT [symbol] TGCG | GATTTTGCG |
| scaffold_6 | 2007502 | AATT [symbol] ATAA | AATT [symbol] ATAA | AATTGATAA | poor depth | poor depth | AATT [symbol] ATAA | AATT [symbol] ATAA | AATT [symbol] ATAA |
| scaffold_7 | 100284 | GAAA [symbol] TCAG | GAAA [symbol] TCAG | GAAA [symbol] TCAG | poor depth | GAAATTCAG | GAAA [symbol] TCAG | GAAA [symbol] TCAG | GAAA [symbol] TCAG |
| scaffold_7 | 348994 | CCG [symbol] GTTT | CCGG [symbol] GTTT | CCGG [symbol] GTTT | CCGG [symbol] GTTT | CCGG [symbol] GTTT | CCGGAGTTT | CCGG [symbol] GTTT | CCGG [symbol] GTTT |
| scaffold_7 | 600111 | CAAT [symbol] ATTA | CAATTATTA | CAAT [symbol] ATTA | CAAT [symbol] ATTA | CAAT [symbol] ATTA | CAAT [symbol] ATTA | CAAT [symbol] ATTA | CAAT [symbol] ATTA |
| scaffold_7 | 850516 | TGAC [symbol] CATA | TGACGCATA | TGAC [symbol] CATA | TGAC [symbol] CATA | TGAC [symbol] CATA | TGAC [symbol] CATA | TGAC [symbol] CATA | TGAC [symbol] CATA |
| scaffold_7 | 873221 | AATA [symbol] ACCT | AATA [symbol] ACCT | AATA [symbol] ACCT | AATA [symbol] ACCT | AATA [symbol] ACCT | AATA [symbol] ACCT | AATA [symbol] ACCT | AATA [symbol] ACCT |
| scaffold_7 | 1100248 | TCAC [symbol] GAAG | TC [symbol] C [symbol] GAAG | TCAC [symbol] GAAG | TCACGGAAG | TCAC [symbol] GAAG | TCAC [symbol] GAAG | TCAC [symbol] GAAG | TCAC [symbol] GAAG |
| scaffold_7 | 1352529 | TAAATATAT | TAAA [symbol] AT [symbol] T | TAAATATAT | [symbol] AAA [symbol] ATAT | [symbol] AAA [symbol] ATAT | TAAA [symbol] AT [symbol] T | TAAATATAT | TAAATATAT |
| scaffold_7 | 1605059 | GACA [symbol] GCAA | GACA [symbol] GCAA | GACAk [symbol] GCAA | GACA [symbol] GCAA | GACA [symbol] CAA | GACAAGCAA | GACA [symbol] GCAA | GACA [symbol] GCAA |
| scaffold_7 | 1991524 | CAAC [symbol] CACC | CAAC [symbol] CACC | CAACCCACC | CAAC [symbol] CACC | CAACCCACC | CAAC [symbol] ACC | CAAC [symbol] CACC | CAAC [symbol] CACC |
| scaffold_8 | 350000 | ATTG [symbol] CGCG | ATTG [symbol] CGCG | ATTG [symbol] CGCG | poor depth | ATTG [symbol] CGCG | ATTG [symbol] CGCG | ATTG [symbol] CGCG | ATTG [symbol] CGCG |
| scaffold_8 | 606991 | GTGT [symbol] TTCT | GTGT [symbol] TTCT | GTGT [symbol] TTCT | GTGT [symbol] TTCT | GTGT [symbol] TTCT | GTGTATTCT | GTGT [symbol] TTCT | GTGT [symbol] TTCT |
| scaffold_8 | 610549 | GGAA [symbol] TTGA | GGAA [symbol] GA | GGAA [symbol] GA | GGAA [symbol] TTGA | GGAA [symbol] TGA | GGAA [symbol] TTGA | GGAA [symbol] GA | GGAA [symbol] GA |
| scaffold_8 | 829832 | CTGT [symbol] CAAC | CTGT [symbol] CAAC | CTGT [symbol] CAAC | CTGTACAAC | CTGTACAAC | CTGTACAAC | CTGT [symbol] CAAC | CTGT [symbol] CAAC |
| scaffold_8 | 829846 | TTCGAGTGA | TTCGAGTGA | TTCGAGTGA | TTCGAGTGA | TTCGAGTGA | TTCG [symbol] GTGA | TTCGAGTGA | TTCGAGTGA |
| scaffold_8 | 830003 | AACTGGCAG | AACT [symbol] GCAG | AACT [symbol] GCAG | AACT [symbol] GCAG | AACT [symbol] GCAG | AACTGGCAG | AACT [symbol] GCAG | AACT [symbol] GCAG |
| scaffold_8 | 830070 | ATTAGGATT | ATTAGGATT | ATTAGGATT | ATTAGGATT | ATTA [symbol] GATT | ATTAGGATT | ATTAGGATT | ATTAGGATT |
| scaffold_8 | 830078 | TACT [symbol] GACG | TACT [symbol] GACG | TACT [symbol] GACG | TACT [symbol] GACG | TACT [symbol] GACG | TACT [symbol] GACG | TACT [symbol] GACG | TACT [symbol] GACG |
| scaffold_8 | 830105 | ATTTAGCAT | ATTTAGCAT | ATTTAGCAT | ATTTAGCAT | ATTTAGCAT | ATTTAGCAT | ATTTAGCAT | ATTTAGCAT |
| scaffold_8 | 830159 | AATTAGAAG | AATTAGAAG | AATTAGAAG | AATTAGAAG | AATTAGAAG | AATT [symbol] GAAG | AATTAGAAG | AATTAGAAG |
| scaffold_8 | 830169 | GACGACTGG | GACGACTGG | GACGACTGG | GACGACTGG | GACGACTGG | GACG [symbol] C [symbol] GG | GACGACTGG | GACGACTGG |
| scaffold_8 | 830215 | AGTG [symbol] ATCT | AGTG [symbol] ATCT | AGTG [symbol] ATCT | AGTG [symbol] ATCT | AGTG [symbol] ATCT | AGTG [symbol] ATCT | AGTG [symbol] ATCT | AGTG [symbol] ATCT |
| scaffold_8 | 830250 | TCCA [symbol] TGCA | TCCA [symbol] TGCA | TCCA [symbol] TGCA | TCCA [symbol] TGCA | TCCA [symbol] TGCA | TCCA [symbol] TGCA | TCCA [symbol] TGCA | TCCA [symbol] TGCA |
| scaffold_8 | 1100000 | CATACGATC | CATACGATC | CATACGATC | CATACGATO | CATACGATC | CATACGATC | CATACGATC | CATACGATC |
| scaffold_8 | 1350240 | ACGG [symbol] TACT | ACGG [symbol] TACT | ACGG [symbol] TACT | ACGGGTACT | ACGGGTACT | ACGGGTACT | ACGG [symbol] TACT | ACGG [symbol] TACT |
| scaffold_8 | 1354068 | AGAA [symbol] GCCT | AGAA [symbol] G [symbol] T | AGAA [symbol] G [symbol] T | AGAA [symbol] G [symbol] T | AGAA [symbol] G [symbol] T | AGAA [symbol] GCCT | AGAA [symbol] G [symbol] T | AGAA [symbol] G [symbol] T |
| scaffold_8 | 1614036 | TTAT [symbol] AGTA | TTAT [symbol] AGTA | TTAT [symbol] AGTA | TTATCAGTA | TTAT [symbol] AGTA | TTAT [symbol] AGTA | TTAT [symbol] AGTA | TTAT [symbol] AGTA |
| scaffold_8 | 1869238 | TGGA [symbol] GTTG | TGGA [symbol] GTTG | TGGA [symbol] GTTG | TGGA [symbol] GTTG | TGGA [symbol] GTTG | TGGA [symbol] GTTG | TGGA [symbol] GTTG | TGGA [symbol] GTTG |
| scaffold_9 | 100447 | CTAT [symbol] TTCT | CTAT [symbol] TTCT | CTAT [symbol] TTCT | CTAT [symbol] TTCT | CTAT [symbol] TTCT | CTATTTTCT | CTAT [symbol] TTCT | CTAT [symbol] TTCT |
| scaffold_9 | 350569 | AGAA [symbol] ATAC | AGAATATAC | AGAA [symbol] ATAC | AGAA [symbol] ATAC | AGAA [symbol] ATAC | AGAATATAC | AGAATATAC | AGAA [symbol] ATAC |
| scaffold_9 | 599950 | T [symbol] GT [symbol] TCCC | TGGT [symbol] TCCC | TGGT [symbol] TCCC | TGGT [symbol] TCCC | TGGT [symbol] TCCC | TGGT [symbol] TCCC | TGGT [symbol] TCCC | TGGT [symbol] TCCC |
| scaffold_9 | 611788 | T [symbol] TG [symbol] ATC | T [symbol] TGTAATC | T [symbol] TG [symbol] ATC | T [symbol] TGTAATC | T [symbol] TGTAATC | T [symbol] TGTAATC | T [symbol] TGTAATC | T [symbol] TGTAATC |
| scaffold_9 | 721973 | TGTA [symbol] ACGT | TGTA [symbol] AC [symbol] T | TGTA [symbol] ACGT | TGTA [symbol] ACGT | TGTA [symbol] ACGT | TGTA [symbol] ACGT | TGTA [symbol] AC [symbol] T | TGTA [symbol] AC [symbol] T |
| scaffold_9 | 1010845 | G [symbol] GTGGTGA | GGGTGGTGA | G [symbol] GT [symbol] GTGA | GGGT [symbol] GTGA | GGGT [symbol] GTGA | GGGTGGTGA | GGGTGGTGA | GGGT [symbol] GTGA |
| scaffold_9 | 1250830 | TTGT [symbol] GGGA | TTGTGGGGA | TTGT [symbol] GGGA | TTGT [symbol] GGGA | TTGT [symbol] GGGA | TTGT [symbol] GGGA | TTGTGGGGA | TTGT [symbol] GGGA |
| scaffold_9 | 1499265 | AGTC [symbol] GACA | AGTC [symbol] GACA | AGTC [symbol] GACA | AGTC [symbol] GACA | AGTC [symbol] GACA | AGTC [symbol] GACA | AGTC [symbol] GACA | AGTC [symbol] GACA |
| scaffold_9 | 1499300 | TATG [symbol] C [symbol] CC | TATGAC [symbol] CC | TATGAC [symbol] CC | TATGAC [symbol] CC | TATGAC [symbol] CC | TATGAC [symbol] CC | TATGAC [symbol] CC | TATGAC [symbol] CC |
| scaffold_9 | 1676755 | CTGC [symbol] GTTT | CTGC [symbol] GTTT | CTGC [symbol] GTTT | CTGC [symbol] GTTT | CTGCCGTTT | CTGC [symbol] GTTT | CTGC [symbol] GTTT | CTGC [symbol] GTTT |
| scaffold_9 | 1702348 | AGAC [symbol] CATC | AGA [symbol] CATC | AGAC [symbol] CATC | AGA [symbol] CATC | AGAC [symbol] CATC | AGAC [symbol] CATC | AGA [symbol] CATC | AGAC [symbol] CATC |
| scaffold_9 | 1702552 | CAAAGTCAT | CAAAGTCAT | CAAAGTCAT | CAAAGTCAT | CAAAGTCAT | CAAAGTC [symbol] T | CAAAGTCAT | CAAAGTCAT |
| scaffold_9 | 1702583 | ACTCAGCTG | ACTCAGCTG | ACTCAGCTG | ACTCAGCTG | ACTCAGCTG | ACTCAGCTG | ACTCAGCTG | ACTCAGCTG |
| scaffold_9 | 1702658 | TTGT [symbol] TGG | TTGT [symbol] TGG | TTGT [symbol] TGG | TTGTC [symbol] TGG | TTGTC [symbol] TGG | TTGT [symbol] GTGG | TTGT [symbol] TGG | TTGT [symbol] TGG |
| scaffold_10 | 100470 | TCAC [symbol] ATCG | TCAC [symbol] ATCG | TCACCATCG | TCACCATCG | TCACCATCG | TCACCATCG | TCACCATCG | TCACCATCG |
| scaffold_10 | 350030 | GCGG [symbol] TCAA | GCGG [symbol] TCAA | GCGG [symbol] TCAA | GCGG [symbol] TCAA | GCGG [symbol] TCAA | GCGG [symbol] TCAA | GCGG [symbol] TCAA | GCGG [symbol] TCAA |
| scaffold_10 | 354531 | AATC [symbol] ATCA | AATC [symbol] ATCA | AATC [symbol] ATCA | AATC [symbol] ATCA | AATC [symbol] ATCA | AATC [symbol] ATCA | AATC [symbol] ATCA | AATC [symbol] ATCA |
| scaffold_10 | 633622 | TGGG [symbol] AAAG | TGGG [symbol] AAAG | TGGG [symbol] AAAG | TGGG [symbol] AAAG | TGGG [symbol] AAAG | TGGG [symbol] AAAG | TGGG [symbol] AAAG | TGGG [symbol] AAAG |
| scaffold_10 | 860249 | CCGC [symbol] AATT | CCGC [symbol] AATT | CCGCAAATT | CCGC [symbol] AATT | CCGC [symbol] AATT | CCGC [symbol] AATT | TGGG [symbol] AAAG | CCGC [symbol] AATT |
| scaffold_10 | 863401 | ATAAAATTT | AT [symbol] AA [symbol] TTT | ATAAAATTT | ATAAAATTT | ATAA [symbol] ATTT | ATAAAATTT | AT [symbol] AA [symbol] TTT | ATAA [symbol] TTT |
| scaffold_10 | 1107782 | CAACCCCAC | CAACCCCAC | CAACCCCAC | poor depth | poor depth | CAAC [symbol] CCAC | CAACCCCAC | CAACmCCAC |
| scaffold_10 | 1338596 | GTGC [symbol] TCAT | GTGC [symbol] TCAT | GTGC [symbol] TCAT | GTGC [symbol] TCAT | GTGC [symbol] TCAT | GTGC [symbol] TCAT | GTGC [symbol] TCAT | GTGC [symbol] TCAT |
| scaffold_10 | 1477092 | AGAT [symbol] CAAA | A [symbol] AT [symbol] CAAA | AGATGCAAA | A [symbol] ATGCAAA | A [symbol] ATG [symbol] A [symbol] A | AGATG [symbol] A [symbol] A | A [symbol] AT [symbol] CAAA | AGATsCAAA |
| scaffold_10 | 1612161 | TCTTCGGAG | TCTTCGGAG | TCTT [symbol] GGAG | TCTT [symbol] GGAG | TCTTCGGAG | TCTTCGGAG | TCTTCGGAG | TCTTCGGAG |
| scaffold_10 | 1612569 | ATTATATTC | ATTATATTC | ATTATATTC | ATTATATTC | ATTATATTC | ATTATATTC | ATTATATTC | ATTATATTC |
| scaffold_10 | 1612630 | TGGCTCCTT | TGGCTCCTT | TGGCTCCTT | TGGCTCCTT | TGGC [symbol] CCTT | TGGC [symbol] CCTT | TGGCTCCTT | TGGC [symbol] CCTT |
| scaffold_10 | 1612671 | GGAATCGTC | GGAATCGTC | GGAATCGTC | GGAATCGTC | GGAA [symbol] CGTC | GGAA [symbol] CGTC | GGAATCGTC | GGAA [symbol] CGTC |
| scaffold_11 | 101855 | CCAG [symbol] CTGT | CCAGCCTGT | CCAGCCTGT | poor depth | CCAGCCTGT | CCAGCCTGT | CCAG [symbol] CTGT | CCAG [symbol] CTGT |
| scaffold_11 | 173230 | AGCGGGCGA | AGCG [symbol] GCGA | AGCG [symbol] GCGA | AGCG [symbol] GCGA | AGCG [symbol] GCGA | AGCG [symbol] GCGA | AGCGGGCGA | AGCGGGCGA |
| scaffold_11 | 350000 | GTCAGCAAG | GTCAGCAAG | GTCAGCAAG | GTCAGCAAG | GTCAGCAAG | GTCAGCAAG | GTCAGCAAG | GTCAGCAAG |
| scaffold_11 | 378409 | TGAT [symbol] GGGG | TGATTGGGG | TGAT [symbol] GGGG | TGAT [symbol] GGGG | TGAT [symbol] GGGG | TGAT [symbol] GGGG | TGATTGGGG | TGAT [symbol] GGGG |
| scaffold_11 | 600001 | TGGG [symbol] GCGC | TGGG [symbol] GCGC | TGGG [symbol] GCGC | TGGG [symbol] GCGC | TGGG [symbol] GCGC | TGGG [symbol] GCGC | TGGG [symbol] GCGC | TGGG [symbol] GCGC |
| scaffold_11 | 627221 | TCTT [symbol] GCCC | TCTT [symbol] GCCC | TCTT [symbol] GCCC | TCTT [symbol] GCCC | TCTT [symbol] GCCC | TCTT [symbol] GCCC | TCTT [symbol] GCCC | TCTT [symbol] GCCC |
| scaffold_11 | 929659 | GGAA [symbol] TCA | GGAA [symbol] TCA | GGAA [symbol] TCA | GGAA [symbol] TCA | GGAA [symbol] TCA | GGAA [symbol] TCA | GGAA [symbol] TCA | GGAA [symbol] TCA |
| scaffold_11 | 931877 | GACC [symbol] CACC | GACC [symbol] CACC | GACC [symbol] CACC | GACC [symbol] CACC | GACC [symbol] CACC | GACC [symbol] CACC | GACC [symbol] CACC | GACC [symbol] CACC |
| scaffold_11 | 1155850 | T [symbol] TG [symbol] CACG | T [symbol] TGCCACG | T [symbol] T [symbol] CCACG | T [symbol] T [symbol] CCACG | T [symbol] T [symbol] CCACG | T [symbol] T [symbol] CCACG | T [symbol] TG [symbol] CACG | TrTryCACG |
| scaffold_11 | 1240230 | ACAA [symbol] ATTC | ACAA [symbol] ATTC | ACAAGATTC | ACAA [symbol] ATTC | ACAAGATTC | ACAAGATTC | ACAA [symbol] ATTC | ACAA [symbol] ATTC |
| scaffold_11 | 1250447 | GAGG [symbol] TACA | GAGG [symbol] TACA | GAGG [symbol] TACA | GAGG [symbol] TACA | GAGG [symbol] TACA | GAGG [symbol] TACA | GAGG [symbol] TACA | GAGG [symbol] TACA |
| scaffold_12 | 109790 | GTCT [symbol] CACC | GTCT [symbol] CACC | GTCT [symbol] CACC | GTCTGCACC | GTCTGCACC | GTCTGCACC | GTCT [symbol] CACC | GTCT [symbol] CACC |
| scaffold_12 | 272255 | CCGA [symbol] TGCT | CCGA [symbol] TGCT | CCGA [symbol] TGCT | CCGA [symbol] TGCT | CCGA [symbol] TGCT | CCGA [symbol] TGCT | CCGA [symbol] TGCT | CCGA [symbol] TGCT |
| scaffold_12 | 281720 | CTTC [symbol] CG | CTTC [symbol] CG | CTTC [symbol] CG | CTTC [symbol] CG | CTTC [symbol] CG | CTTC [symbol] CG | CTTC [symbol] CG | CTTC [symbol] CG |
| scaffold_12 | 281763 | TCTG [symbol] AGCC | TCTG [symbol] AGCC | TCTG [symbol] AGCC | TCTGCAGCC | TCTGCAGCC | TCTGCAGCC | TCTG [symbol] AGCC | TCTGCAGCC |
| scaffold_12 | 554582 | ACTC [symbol] GGTC | ACTC [symbol] GGTC | ACTC [symbol] GGTC | ACTC [symbol] GGTC | ACTC [symbol] GGTC | ACTCCGGTC | ACTC [symbol] GGTC | ACTCAGGTC |
| scaffold_12 | 770075 | GAAC [symbol] TTCT | GAAC [symbol] TTCT | GAAC [symbol] TTCT | GAAC [symbol] TTCT | GAAC [symbol] TTCT | GAAC [symbol] TTCT | GAAC [symbol] TTCT | GAAC [symbol] TTCT |
| scaffold_12 | 909536 | CTAT [symbol] GAGG | CTAT [symbol] GAGG | CTAT [symbol] GAGG | CTATGGAGG | CTAT [symbol] AGG | CTAT [symbol] AGG | CTAT [symbol] GAGG | CTATGGAGG |
| scaffold_12 | 1000000 | CGAG [symbol] AGGA | CGAG [symbol] AGGA | CGAG [symbol] AGGA | poor depth | CGAG [symbol] AGGA | CGAG [symbol] AGGA | CGAG [symbol] AGGA | CGAG [symbol] AGGA |
| scaffold_13 | 100697 | ACGTCTTTA | ACGTCTTTA | ACGTCTTTA | ACGT [symbol] TTTA | ACGTCTTTA | ACGT [symbol] TTTA | ACGTCTTTA | ACGTCTTTA |
| scaffold_13 | 119283 | ACG [symbol] ACTG | ACG [symbol] ACTG | ACGTTACTG | poor depth | ACG [symbol] ACTG | ACG [symbol] ACTG | ACG [symbol] ACTG | ACG [symbol] ACTG |
| scaffold_13 | 363867 | ATCC [symbol] CTGC | ATCC [symbol] CTGC | ATCCACTGC | ATCC [symbol] CTGC | ATCC [symbol] CTGC | ATCC [symbol] CTGC | ATCC [symbol] CTGC | ATCC [symbol] CTGC |
| scaffold_13 | 370521 | TTTG [symbol] GTCA | TTTG [symbol] GTCA | TTTGAGTCA | TTTGAGTCA | TTTG [symbol] GTCA | TTTGAGTCA | TTTG [symbol] GTCA | TTTGTGTCA |
| scaffold_13 | 604345 | CTTCAGCAT | CTTCAGCAT | CTTCAGCAT | CTTC [symbol] GCAT | CTTCAGCAT | CTTCAGCAT | CTTCAGCAT | CTTCAGCAT |
| scaffold_13 | 866136 | GTTG [symbol] TCAG | GTTG [symbol] TCAG | GTTGGTCAG | poor depth | GTTG [symbol] TCAG | GTTG [symbol] TCA [symbol] | GTTG [symbol] TCAG | GTTG [symbol] TCAG |
| scaffold_14 | 113109 | AGGG [symbol] AATA | AGGG [symbol] AATA | AGGG [symbol] AATA | AGGG [symbol] AATA | AGGG [symbol] AATA | AGGG [symbol] AATA | AGGG [symbol] AATA | AGGG [symbol] AATA |
| scaffold_14 | 372086 | CGAT [symbol] CCTT | CGAT [symbol] CCTT | CGAT [symbol] C [symbol] TT | CGAT [symbol] C [symbol] TT | CGAT [symbol] C [symbol] TT | CGAT [symbol] C [symbol] TT | CGAT [symbol] CCTT | CGAT [symbol] CCTT |
| scaffold_14 | 603118 | GGCC [symbol] GCCT | GGCC [symbol] GCCT | GGCCSGCCT | GGCCCGCCT | GGCCCGCCT | GGCCCGCCT | GGCC [symbol] GCCT | GGCC [symbol] GCCT |
| scaffold_14 | 725687 | AGTT [symbol] G [symbol] AA | AGTT [symbol] G [symbol] AA | AGTT [symbol] G [symbol] AA | A [symbol] TT [symbol] GAAA | A [symbol] TT [symbol] GAAA | A [symbol] TT [symbol] G [symbol] AA | AGTT [symbol] G [symbol] AA | A [symbol] TT [symbol] GAAA |
| scaffold_14 | 808308 | AAG [symbol] ATGG | AAG [symbol] ATGG | AAGGTATGG | poor depth | AAGG [symbol] ATGG | AAGG [symbol] ATGG | AAG [symbol] ATGG | AAG [symbol] ATGG |
| scaffold_15 | 101381 | TAAA [symbol] AGAT | TAAA [symbol] AGAT | TAAACAGAT | poor depth | TAAA [symbol] AGAT | TAAACAGAT | TAAA [symbol] AGAT | TAAA [symbol] AGAT |
| scaffold_15 | 150013 | GTGG [symbol] CCGT | GTGG [symbol] CCGT | GTGGCCCGT | GTGGCCCGT | GTGGCCCGT | GTGGCCCGT | GTGG [symbol] CCGT | GTGG [symbol] CCGT |
| scaffold_15 | 367204 | CGCG [symbol] CCTA | CGCG [symbol] CCTA | CGCGCCCTA | CGCG [symbol] CCTA | CGCG [symbol] CCTA | CGCG [symbol] CCTA | CGCG [symbol] CCTA | CGCG [symbol] CCTA |
| scaffold_16 | 106292 | AAGC [symbol] GGAA | AAGC [symbol] GGAA | AAGCTGGAA | AAGC [symbol] GGAA | AAGCTGGAA | AAGC [symbol] GGAA | AAGC [symbol] GGAA | AAGC [symbol] GGAA |
| scaffold_16 | 205778 | CAAG [symbol] TCTG | CAAG [symbol] TCTG | CAAG [symbol] TCTG | CAAG [symbol] TCTG | CAAG [symbol] TCTG | CAAG [symbol] TCTG | CAAG [symbol] TCTG | CAAG [symbol] TCTG |
| scaffold_16 | 400000 | CCTCGGATT | CCTCGGATT | CCTCGGATT | CCTCGGATT | CCTCGGATT | CCTCGGATT | CCTCGGATT | CCTCGGATT |
| scaffold_16 | 403998 | CAAA [symbol] TACG | CAAAGTACG | CAAA [symbol] TACG | poor depth | CAAA [symbol] TACG | CAAA [symbol] TACG | CAAA [symbol] TACG | CAAA [symbol] TACG |
| scaffold_17 | 134688 | CCCG [symbol] TTCA | CCCG [symbol] TTCA | CCCG [symbol] TTCA | CCCGCTTCA | CCCGCTTCA | CCCGCTTCA | CCCG [symbol] TTCA | CCCGCTTCA |
| scaffold_17 | 370858 | GACA [symbol] AACG | GACAyAACG | GACA [symbol] AACG | GACA [symbol] AACG | GACACAACG | GACACAACG | GACA [symbol] AACG | GACA [symbol] AACG |
| scaffold_17 | 449833 | ATCA [symbol] AC [symbol] A | ATCA [symbol] AC [symbol] A | ATCA [symbol] AC [symbol] A | ATCAGACAA | ATCAGACAA | ATCA [symbol] AC [symbol] A | ATCA [symbol] AC [symbol] A | ATCA [symbol] AC [symbol] A |
| scaffold_17 | 472545 | CCGT [symbol] C [symbol] TG | CCGT [symbol] C [symbol] TG | CCGT [symbol] C [symbol] TG | CCGTTCATG | CCGT [symbol] C [symbol] TG | CCG [symbol] TCA [symbol] G | CCGT [symbol] C [symbol] TG | CCGT [symbol] C [symbol] TG |
| scaffold_18 | 112940 | GCGG [symbol] TGGG | GCGG [symbol] TGGG | GCGGGTGGG | GCGGGTGGG | GCGGGTGGG | GCGGGTGGG | GCGGGTGGG | GCGG [symbol] TGGG |
| scaffold_18 | 126322 | CCTC [symbol] TCCG | CCTC [symbol] TCCG | CCTC [symbol] TCCG | CCTC [symbol] TCCG | CCTC [symbol] TCCG | CCTC [symbol] TCCG | CCTC [symbol] TCCG | CCTC [symbol] TCCG |
| scaffold_19 | 87323 | CCCA [symbol] GCAA | CCCA [symbol] GCAA | CCCA [symbol] GCAA | CCCA [symbol] GCAA | CCCA [symbol] GCAA | CCCA [symbol] GCAA | CCCA [symbol] GCAA | CCCA [symbol] GCAA |
| scaffold_19 | 98782 | AAAA [symbol] TGTT | AAAA [symbol] TGTT | AAAA [symbol] TGTT | AAAA [symbol] T [symbol] TT | AAAA [symbol] T [symbol] TT | AAAATTGTT | AAAA [symbol] TGTT | AAAA [symbol] TGTT |

The use of these markers to determine calculated % genetic similarity (identity) between two heterokaryotic cultures or strains is presented in Table VI.

The 9-mers containing the reported SNPs in the tables have been treated as composites of two unitary alleles (in heterokaryon comparisons).

The composite 9-mer genotype has been compared at each locus and assigned a value if 1 for a perfect match, or a 0 for anything less than a perfect match. Then the values were totaled for all loci in each pairwise comparison between strains, and divided by the total number of loci compared, and the resulting decimal was converted to %.

TABLE VI
Calculation of % similarity (identity) between strain
LA3782 and seven other heterokaryotic strains

| Comparison | Heirloom/ | Tuscan/ | | | | | |
|---|---|---|---|---|---|---|---|
| w/LA3782 | BR06 | B14528 | S-600 | Bs526 | Fr 24 | Brawn | J15051 |
| Similarity | 54% | 57% | 35% | 26% | 25% | 59% | 67% |
| (N = 203) | | | | | | | |
| Similarity | 51% | 55% | 30% | 22% | 23% | 57% | 70% |
| (N = 170) | | | | | | | |

Results are similar for the full set of SNP markers (N=203) and for a smaller set (N=170) excluding the SNPs that define alleles at the six SCAR marker loci (and which have a shorter interval distribution). The highest % genetic similarity or identity observed for LA3782 compared to the heterokaryotic genotypes of seven other strains is 67%. Identity for two clones of LA3782 would be 100%.

B. Vegetative Incompatibility

Substantial genetic dissimilarity (i.e., 100%−% genetic identity) is known to be associated with heterokaryon or ‘vegetative’ incompatibility. Incompatibility interferes with anastomosis and with mushroom production. From the data in Table VI, it would be expected that LA3782 would be incompatible with the other leading commercial brown-capped Agaricus bisporus strains. Table VII demonstrates this empirically.

TABLE VII
Vegetative incompatibility between LA3782, Tuscan and Heirloom:
Numbers of harvested mushrooms after 16 days of cultivation.

| | | | Strain in the compost | | |
|---|---|---|---|---|---|
| | | | LA3782 | HRLM | Tuscan |
| Strain | LA3782 | a | 5 | 0 | 0 |
| In | | b | 4 | 0 | 0 |
| the | | c | 2 | 0 | 0 |
| casing | | | Compatible | Incompatible | Incompatible |
| | | p value | | <0.0001 | 0.001 |
| | Heirloom/ | a | 0 | 1 | 0 |
| | BR06 | b | 0 | 1 | 0 |
| | | c | 0 | 1 | 0 |
| | | | Incompatible | Compatible | Incompatible |
| | | p value | 0.014 | | 0.001 |
| | Tuscan/ | a | 0 | 0 | 2 |
| | B14528 | b | 0 | 0 | 3 |
| | | c | 0 | 0 | 3 |
| | | | Incompatible | Incompatible | Compatible |
| | | p value | 0.014 | <0.0001 | |

General t-test analysis on three replicates (a-c); the difference between compatible and incompatible combinations is significant with p-value a ≤0.05 in all cases. In each treatment, one of the three strains was inoculated into compost, and after colonization, casing soil inoculated with one of the three strains was applied over the compost. Cultivation containers with 0.07 square meters of surface were used in standard growing conditions. Note that only combinations scored as compatible (marked with *) produced mushrooms.

C. Crop Yield

Yield performance was measured in large-scale trials. During these trials, incubation period was 18 days in bulk phase III tunnel, spawning rate was 8 litres/ton of compost phase II. Trays were filled with 135 kg incubated compost with a filling rate of 90 kg/m². Mc substrate supplement was added at the rate of 1.33 kg/m². Carbo 9 casing from supplier Euroveen was applied with 1200 g/m² compost casing, premixed. In the growing room we tested strains with 12 replications distributed across 5 growing levels.

Airing started on day 4 after casing. To collect yield, mushrooms were picked and weighted daily on 12 replicates. Data were collected over 3 flushes.

The mushroom crop yield of strain LA3782 was found to be greater (better) than that of the BR06/Heirloom strain on third flush and also when aggregated over flushes 1, 2 and 3, as shown in Table VIII.

TABLE VIII
Yield comparisons of LA3782 with Heirloom, Tuscan and J15051 strains

| | | 1st flush | 2nd Flush | 3rd flush | Total |
|---|---|---|---|---|---|
| LA3782 | Yield | 17.4 | 12.5 | 9.4 | 39.4 |
| | sd | 1.06 | 1.06 | 1.23 | 1.98 |
| Heirloom/ | Yield | 17.3 | 11.7 | 6 | 34.9 |
| BR06 | sd | 0.72 | 1.78 | 0.9 | 2.4 |
| | p value | 0.66 | 0.17 | < 0.0001 | < 0.0001 |
| Tuscan/ | Yield | 16.5 | 12.9 | 9.3 | 38.6 |
| B14528 | sd | 1.44 | 1.99 | 0.61 | 3.01 |
| | p value | 0.08 | 0.60 | 0.83 | 0.53 |
| J15051 | Yield | 15.0 | 12.1 | 6.2 | 33.3 |
| | sd | 2.34 | 1.46 | 3.01 | 6.80 |
| | p value | 0.51 | 0.003 | 0.835 | 0.141 |

Flush yield and cumulative crop yields of LA3782, Heirloom, Tuscan and J15051 after 1; 2 and 3 flushes, expressed in kg/m2. Standard cultivation and harvest procedures were used. General t-test analysis: the difference with LA3782 is significant at p-value ≤0.05.

From Table VIII, strain LA3782 is shown to be highly productive, and also to have an improved flush-yield balance due to the higher third-flush yield, as compared to all the prior art strains that have been tested.

D. Weight Retention

The mushrooms produced by strain LA3782 also have improved weight retention during post-harvest storage, compared to those of the Heirloom strain, as shown in Table IX.

Trait data collection was carried out by a method in which mushroom samples were collected on the day of peak harvest during a ‘flush’ of mushroom production. A flush lasts four or five days, often with peak production on the second day; typically, three flushes occur at weekly intervals. The expression of the trait in Flush 1 was evaluated. During this test, five replicate styrofoam tills per strain were evaluated. A till is a tray that can hold over 1 kg. The weight of the empty till was recorded. Thirty mushrooms approximately 4-5 cm in diameter, with tightly closed veils were placed into each till. They were spaced enough to not touch each other and placed with the stem up, they were immediately weighed. An initial weight was recorded. The tills were placed at 4° C. for 8 days in a walk-in cooler. Filled till weights were recorded each day beginning on day 3. After subtracting the weight of the empty till, percentage of weight retention was calculated as described above.

TABLE IX
Percentage of initial weight retained after
3-8 days of post-harvest storage at 4° C.

| | % of | % of | % of | % of | % of | % of |
|---|---|---|---|---|---|---|
| | weight | weight | weight | weight | weight | weight |
| Strain | at D3 | at D4 | at D5 | at D6 | at D7 | at D8 |
| LA3782 | 90.2% | 87.6% | 84.5% | 81.4% | 78.6% | 75.4% |
| LA3782 | 91.5% | 89.2% | 87.1% | 84.7% | 82.7% | 79.8% |
| LA3782 | 92.2% | 90.0% | 87.9% | 85.5% | 83.4% | 80.4% |
| LA3782 | 92.2% | 90.0% | 87.5% | 85.6% | 83.6% | 80.4% |
| LA3782 | 90.6% | 88.2% | 85.5% | 83.2% | 80.5% | 77.3% |
| Average | 91.3% | 89.0% | 86.5% | 84.1% | 81.8% | 78.7% |
| HRLM | 87.5% | 83.5% | 80.4% | 76.8% | 73.9% | 70.2% |
| HRLM | 87.9% | 84.3% | 81.3% | 78.1% | 75.3% | 71.9% |
| HRLM | 88.0% | 84.3% | 81.0% | 77.8% | 75.1% | 71.7% |
| HRLM | 88.1% | 84.5% | 81.2% | 78.0% | 75.3% | 72.1% |
| HRLM | 88.7% | 85.2% | 82.0% | 79.1% | 76.6% | 73.5% |
| Average | 88.1% | 84.3% | 81.2% | 78.0% | 75.2% | 71.9% |
| p value | <0.0001 | <0.0001 | <0.0001 | 0.0001 | 0.0002 | 0.0003 |
| Tuscan | 89.5% | 86.5% | 82.7% | 79.9% | 77.3% | 74.3% |
| Tuscan | 88.8% | 86.0% | 82.9% | 80.0% | 77.4% | 74.2% |
| Tuscan | 89.1% | 86.5% | 83.5% | 80.5% | 78.1% | 74.6% |
| Tuscan | 90.0% | 87.8% | 85.3% | 82.9% | 80.6% | 77.3% |
| Tuscan | 90.3% | 88.1% | 85.8% | 83.7% | 81.5% | 78.6% |
| Tuscan | 90.3% | 88.1% | 85.8% | 83.7% | 81.5% | 78.6% |
| Average | 89.7% | 87.3% | 84.7% | 82.1% | 79.8% | 76.7% |
| p value | 0.007 | 0.012 | 0.026 | 0.043 | 0.061 | 0.07 |

E. Mushroom Piece Weight

Table X shows that the piece weight (mean individual harvested mushroom weight) in crops from LA3782 is significantly greater than that of the Heirloom or Tuscan strains, especially in first flush. A greater piece weight can reduce the costs of harvesting the crop.

Trait data collection was carried out by a method in which mushroom samples were collected during the first and second flush of mushroom production. The expression of the trait in Flush 1 and Flush 2 was evaluated. During this test, 20 replicate medium size mushrooms (4-5 cm in diameter) per strain over 4 different levels were evaluated. Each replicate was individually weighed.

TABLE X
Weights of individual mushrooms harvested (i.e., piece weight)

| | 1st Flush | | | 2nd flush | | |
|---|---|---|---|---|---|---|
| | Piece | | p | Piece | | p |
| | weight(g) | sd | value | weight (g) | sd | value |
| LA3782 | 35.87 | 7.91 | / | 34.8 | 9.54 | / |
| HRLM | 29.33 | 8.27 | 0.009 | 33.0 | 8.27 | 0.56 |
| Tuscan | 29.25 | 3.17 | 0.002 | 29.5 | 4.07 | 0.04 |

Average piece weight of category medium size mushrooms in Flush 1 and flush 2 expressed in grams. General t-test analysis: the difference with LA3782 is significant with p-value < α = 0.05

The average piece weight of mushrooms in flush 1 and flush 2 expressed in grams. In a general t-test analysis, the differences with LA3782 were significant at a p a ≤0.05 threshold.

F. Cap Color

The mushroom color was measured using a Minolta Chroma Meter CR-200 (mfd. Japan). Sample sizes of thirty medium sized mushrooms at commercial maturity (with closed veils) were harvested from the tests and measured to obtain values for the L*a*b parameters. The Chroma Meter readings were randomly taken at the tops of the mushroom caps. In the L*a*b system, “L” is a brightness variable with 0 representing complete darkness and 100 representing complete whiteness and “b” value represents blueness (−300)/yellowness (+299). In other words, the darker a mushroom cap color, the lower the L value, and the more yellow a mushroom cap color, the higher b value.

TABLE XI
Chromameter value L, a, b of LA3782,
Heirloom and Tuscan strains

| | L Value | sd | a value | sd | b value | sd |
|---|---|---|---|---|---|---|
| LA3782 | 71.49 | 2.9 | 7.12 | 1.02 | 23.28 | 1.28 |
| Heirloom/BR06 | 63.33 | 2.89 | 9.18 | 0.62 | 23.86 | 1.1 |
| Tuscan/B14528 | 65.71 | 3.34 | 8.57 | 0.87 | 25.1 | 0.93 |

Finally, it will be understood that any variations evident fall within the scope of the claimed invention and thus, the specific selection of characteristics, techniques, and sources of homokaryons and heterokaryons can be determined without departing from the spirit of the present invention herein disclosed and described. Further, it will be understood that the scope of the invention is not necessarily limited to methods that produce mushroom strains and cultures that have all of the characteristics set forth herein, but rather to those strains, lines, and cultures that are produced, descended or otherwise derived from cultures having at least one parent that is derived from line N-s34 or strain LA3782. Accordingly, the scope of the invention shall include all modifications and variations that may fall within the scope of the attached claims.

1.-19. (canceled)

20. An Agaricus bisporus culture comprising a complete set of chromosomes of Agaricus bisporus line N-s34, a representative culture of said line N-s34 having been deposited under CNCM Accession Number I-5528, wherein said set of chromosomes comprises the sequence-characterized allelic markers listed in Table I.

21. The Agaricus bisporus culture of claim 20, wherein said culture is selected from the group consisting of:
(a) the line N-s34, and
(b) F1 hybrid strains produced by mating the line N-s34 to a second line.

22. The Agaricus bisporus culture of claim 20, wherein said culture comprises F1 hybrid strains produced by mating the line N-s34 to a homokaryon obtained from strain BP-1, a representative culture of said strain BP-1 having been deposited under ATCC Accession Number PTA-6903.

23. The Agaricus bisporus culture of claim 20, wherein said culture comprises strain LA3782, a representative culture of said strain LA3782 having been deposited under CNCM Accession Number I-5527.

24. An Agaricus bisporus culture comprising a haploid set of chromosomes of strain LA3782, a representative culture of said strain LA3782 having been deposited under CNCM Accession Number I-5527, said haploid set of chromosomes comprising the sequence-characterized allelic markers listed in Table II, provided that said culture is not strain BP-1, a representative culture of said strain BP-1 having been deposited under ATCC Accession Number PTA-6903.

25. The Agaricus bisporus culture of claim 24, wherein said culture is selected from the group consisting of:
(a) a homokaryon of the strain LA3782, and
(b) F2 hybrids produced by mating said homokaryon of (a) with a second line.

26. An Agaricus bisporus culture of F2, F3, F4, or F5 generation descended from F1 hybrids produced by mating a first parent which is line N-s34, a representative culture of said line N-s34 having been deposited under CNCM Accession Number I-5528, with a second parent;
wherein said second parent is optionally strain LA3782 or a strain derived therefrom, a representative culture of strain LA3782 having been deposited under CNCM Accession Number I-5527,
wherein said F2 generation comprises at least 40-60% of the Single-Nucleotide Polymorphisms (SNPs) present in the genome of the strain LA3782;
wherein said F3 generation comprises at least 20-30% of the SNPs present in the genome of the strain LA3782;
wherein said F4 generation comprises at least 10-15% of the SNPs present in the genome of the strain LA3782; and
wherein said F5 generation comprises at least 4-8% of the SNPs present in the genome of the strain LA3782.

27. The Agaricus bisporus culture of claim 26, wherein said second parent is the strain LA3782 or a strain derived therefrom; and wherein said culture comprises at least about 100 allelic markers of the sequence-characterized allelic markers listed in Table II, at least about 50 allelic markers of the sequence-characterized allelic markers in Table II, or at least about 25 of the sequence-characterized allelic markers in Table II.

28. The Agaricus bisporus culture of claim 26, wherein said second parent is the strain LA3782 or a strain derived therefrom, and wherein said culture comprises at least 40-60%, at least 20-30%, at least 10-15%, or at least 4-8% of the sequence-characterized allelic markers listed in Table II or Table III.

29. An Agaricus bisporus culture that is derived from an initial culture, wherein said initial culture is selected from the group consisting of:
a) strain LA3782, a representative culture of said strain LA3782 having been deposited under CNCM Accession Number I-5527,
b) line N-s34, a representative culture of said line N-s34 having been deposited under CNCM Accession Number I-5528.

30. The Agaricus bisporus culture of claim 29, wherein said culture comprises at least 65% of the sequence-characterized allelic markers of the line N-s34 listed in Table I or 65% of the sequence-characterized allelic markers of the strain LA3287 listed in Table II.

31. A composition comprising cells, hyphae, mycelium, mushrooms, germinated spores, ungerminated spores, homokaryons, heterokaryons, and/or aneuploids obtained from the culture of claim 20.

32. A composition comprising cells, hyphae, mycelium, mushrooms, germinated spores, ungerminated spores, homokaryons, heterokaryons, and/or aneuploids obtained from the culture of claim 24.

33. A composition comprising cells, hyphae, mycelium, mushrooms, germinated spores, ungerminated spores, homokaryons, heterokaryons, and/or aneuploids obtained from the culture of claim 26.

34. A composition comprising cells, hyphae, mycelium, mushrooms, germinated spores, ungerminated spores, homokaryons, heterokaryons, and/or aneuploids obtained from the culture of claim 29.

35. A method for developing a new Agaricus bisporus culture, said method comprising applying at least one mushroom strain development technique to the culture of claim 20, wherein said new Agaricus bisporus culture comprises the line N-s34, a homokaryon of strain LA3782, a representative culture of said strain LA3782 having been deposited under the CNCM Accession Number I-5527, or a progeny thereof.

36. The method of claim 36, wherein said new Agaricus bisporus culture is characterized in that:
(a) yield performance of crops produced from said new Agaricus bisporus culture is equal to or exceeds yield performance of crops of a BR06/Heirloom strain of Agaricus bisporus, and
(b) a third-flush yield of crops produced from said new Agaricus bisporus culture exceeds that of the BR06/Heirloom strain, and
(c) mushroom product of crops produced from said new Agaricus bisporus culture retains more weight after a number of days of post-harvest storage at 4 degrees Celsius than does mushroom product of the BR06/Heirloom strain, the number of days selected from the group comprising 3, 4, 5, 6, 7, and 8 days.

37. The method of claim 36, wherein said new Agaricus bisporus culture is an F2, F3, F4, or F5 generation descended from the strain LA3782, or from a strain derived from the strain LA3782, and comprising respectively at least 40-60%, at least 20-30%, at least 10-15%, or at least 4-8% of the Single-Nucleotide Polymorphisms (SNPs) present in the genome of the strain LA3782.

38. The method of claim 36, wherein said new Agaricus bisporus culture comprises at least about 100 allelic markers of the sequence-characterized allelic markers listed in Table II, at least about 50 allelic markers of the sequence-characterized allelic markers listed in Table II or at least about 25 of the sequence-characterized allelic markers listed in Table II.

39. The method of claim 36, wherein said new Agaricus bisporus culture comprises at least 40-60%, at least 20-30%, at least 10-15%, or at least 4-8% of the sequence-characterized allelic markers listed in Table II or Table III.


SEQUENCE LISTING (12 sequences)

SEQ ID NO: 1 - DNA, 18 bp, artificial
  p1n150-3G-2 forward primer
aggcrycccatcttcasc

SEQ ID NO: 2 - DNA, 17 bp, artificial
  p1n150-3G-2 reverse primer
gttcgacgacggactgc

SEQ ID NO: 3 - DNA, 19 bp, artificial
  ITS1 primer
tccgtaggtgaacctgcgg

SEQ ID NO: 4 - DNA, 20 bp, artificial
  ITS4 primer
tcctccgcttattgatatgc

SEQ ID NO: 5 - DNA, 23 bp, artificial
  MFPC-1-ELF forward primer
aytcrcaamaacataccttcaac

SEQ ID NO: 6 - DNA, 18 bp, artificial
  MFPC-1-ELF reverse primer
cattcggcgattttctca

SEQ ID NO: 7 - DNA, 21 bp, artificial
  AN forward primer
gacgatgcgggactggtggat

SEQ ID NO: 8 - DNA, 23 bp, artificial
  AN reverse primer
ggtctggcctacrggagtgttgt

SEQ ID NO: 9 - DNA, 23 bp, artificial
  AS forward primer
ccgccagcacaaggaatcaaatg

SEQ ID NO: 10 - DNA, 23 bp, artificial
  AS reverse primer
tcagtcggccctcaaaacagtcg

SEQ ID NO: 11 - DNA, 22 bp, artificial
  FF forward primer
tcgggtggttgcaactgaaaag

SEQ ID NO: 12 - DNA, 24 bp, artificial
  FF reverse primer
ttcctttccgccttaattgtttct